(19) United States
(12) Patent Application Publication
(10) Pub. No.: US 2018/0279916 A1
(43) Pub. Date: Oct. 4, 2018

(54) System and Method for Monitoring the Running Technique of a User

(71) Applicant: MAS INNOVATION (PVT) LIMITED, Colombo (LK)

(72) Inventors: Jonathan Folland, Leicestershire (GB); Matthew Black, Leicestershire (GB); Joseph Handsaker, (LK); Sam Allen, (GB); Stephanie Forrester, (GB)

(73) Assignee: MAS INNOVATION (PVT) LIMITED, Colombo (LK)

(21) Appl. No.: 15/764,851

(22) PCT Filed: Aug. 2, 2016
(86) PCT No.: PCT/GB2016/052377

§ 371 Date: Mar. 29, 2018

(30) Foreign Application Priority Data
Oct. 2, 2015 (GB) 1517400.6
Apr. 28, 2016 (GB) 1607412.2

(51) Int. Cl. A61B 5/11; A61B 5/00

(52) U.S. Cl. CPC A61B 5/112; A61B 5/1121; A61B 2503/10; A61B 5/486; A61B 5/1118; A61B 5/6804

(57) ABSTRACT

A system and method for monitoring the running technique of a user undertaking a physical activity is described. The system comprises: a garment worn by the user incorporating a sensor for the detection of a parameter relating to the movement of the pelvis of the user; a processing unit configured to receive information about the parameter from the sensor, to compare the parameter with an aspect of a biomechanical model, and to determine if a feedback response is required; and means for providing the feedback response to the user. The method comprises the steps of: measuring a parameter relating to the movement of the pelvis of the individual; comparing the parameter with a biomechanical model to determine whether a feedback response is required and providing a feedback response if required.

This invention relates to systems and methods for monitoring the running technique of a user undertaking a physical activity and to garments suitable for use in such systems and methods. More particularly, the invention relates to systems and methods which provide feedback to the user, for example during the physical activity, to garments suitable for use in such systems and methods, and to the use of kinematic variables and biomechanical models for the monitoring, assessing and improving the running technique of an individual undertaking a physical activity.

There is a significant proportion of the population who regularly undertake an exercise activity. For example, there are 9 million core recreational runners in the US, who typically participate in multiple races each year. Often runners rely on an instinctive feel for their own physiological measures affecting running, as well as for nutrition and conditioning through years of running experience. But they have minimal opportunity to identify whether they are running with correct technique which can be crucial to achieving an enhanced performance as well as to prevent and/or to reduce the likelihood of injuries.

Usually runners or participants in other exercise activities do not have access to a personal coach to observe and advise, and even if they do, typically the coach is not present at all stages of the activity. For example, runners do not often run with their coach, and therefore are unable to obtain advice as to their running technique at the crucial stages of a run when they are fatigued and likely to lose form.

Running performance is known to be influenced by a range of anthropometric, physiological and biomechanical factors, with the latter including running technique which appears to vary widely, particularly at a recreational level. Running technique can be assessed with whole body 3-D motion analysis to determine kinematics of the individual body segments and the whole body centre of mass (CM). Whilst there is extensive coaching opinion on optimal running technique, there is very little objective information available about the ideal technique/kinematics for running performance. In one of the strongest studies performed to date, Williams and Cavanagh (1987) found no kinematic variables to be related to performance, likely due to the small cohort for performance data (n=16) and relatively narrow range of performance times (10 km, 30:36 to 38:30).

Distance running performance is dependent on the velocity of running that can be sustained for the duration of an event. This velocity is determined by the maximum rate at which aerobic energy production can be maintained, which in turn depends on maximal oxygen uptake (\dot{V}O₂max) and lactate threshold; and the efficiency with which this energy can be converted into anterior movement of the CM, known as running economy (RE). The combination of \dot{V}O₂max and RE, has been found to account for ˜94% of the inter-individual variance in running performance over 16.1 km (McLaughlin et al. 2010). Differences in running technique are expected to influence performance through changes to running economy, which can be accurately measured as the energy cost (E_{c}) of running during treadmill tests (Saunders et al., 2004; Shaw et al., 2014; Shaw et al., 2015). However the relationship between specific kinematic variables and RE remains opaque.

It is known to incorporate sensors into exercise equipment and clothing, for example to detect motion of the user. The data collected during a physical activity is typically uploaded by the user after completion of the physical activity for analysis, for example to determine the distance run or the number of steps taken, or may be analysed by remote monitoring by a third party. Such systems typically use for comparison previous data gathered on the user, for example, previous numbers of steps taken, or may utilise a comparison with other users of a system, for example, a comparison of heart rate, steps taken etc. over a running route. These systems typically do not provide any information relevant to a user's technique, and therefore are not able to help to improve running performance.

US2013/0190658A1 (Myotest S A) describes a system and method for detecting asymmetries in the movement of a user. The system involves fastening a device on the torso of a user and measuring acceleration data relating to the movement of the user's centre of mass.

There is a need for the provision of enhanced systems and methods which enable feedback to a user on his or her running technique and which can therefore help to improve running performance and to prevent and/or to reduce injury.

### SUMMARY OF THE INVENTION

In a first aspect of the invention, there is provided a system for monitoring the running technique of a user undertaking a physical activity, the system comprising at least one garment worn by the user, the garment incorporating or carrying at least one sensor for the detection of at least one parameter relating to the motion of the user; a processing unit configured to receive information about the at least one parameter from the at least one sensor, to compare the or each parameter with at least one aspect of a biomechanical model of the physical activity, and to determine if a feedback response is required; and means for providing the feedback response to the user.

The system of the invention enables the user to receive feedback on his or her running technique whilst undertaking a physical activity, such as distance running, or other sporting activities that involve the individual running, such as football, field hockey, rugby, lacrosse, orienteering, etc., and to receive feedback. The feedback provided enables the user to enhance their technique, for example relating to their running form, such as knee positioning, hip positioning, stride length etc., or a combination of these factors. Preferably, the feedback response is provided to the user during the physical activity. Maintaining and/or improving technique during a physical activity can help to enhance performance, for example enhance endurance and/or speed and reduce the likelihood of injuries.

The system incorporates at least one garment, such as a running garment, which is worn by the user, which could be, for example, a pair of shorts, a vest, a t-shirt, training top, leggings, etc. The garment is typically a base layer, or other body fitting apparel. Preferably the garment is close fitting to the body of the user, for example close fitting to the torso, arms, legs etc. or any combination of these, preferably close fitting to the torso. The system may incorporate a combination of garments, for example a combination of a garment worn on the lower half of the user's body and a garment worn on the upper half, such as a pair of shorts or leggings and a t-shirt or training top. This enables sensors to be placed in positions to monitor motion in the both the upper body, such as the arms, and lower body, such as the legs. In one embodiment, the garment is not a foot-receiving garment, such as a sock.

The garment incorporates at least one sensor which detects at least one parameter relating to the motion of the user. Such parameters may relate, for example, to the speed, direction of movement, and/or acceleration of at least one part of the body of the user or to the relative speed, direction or movement and/or acceleration of two or more parts of the body, or other kinematic data. The sensor may, for example, be an accelerometer or a gyroscope. The system may use a combination of sensor types, for example a combination of at least one accelerometer and at least one gyroscope. The use of at least one sensor incorporated within a garment provides more accurate data at a specific body location rather than an approximation. For the detection of parameters relating to the movement of the pelvis, it is preferable to place one or more sensors close to the pelvic region, such as in the waistband region of a garment. It will be understood that the or each parameter may be measured over the course of a single step and/or a single stride, or may be measured over a multiplicity of strides and, for example, the average value assessed.

It has been found that the analysis of the motion of the pelvis whilst an individual is running can have high utility in the assessment of running technique. For example, it has been found that the minimum velocity of the pelvis and the change in vertical position of the pelvis are correlated with the energy cost of running and velocity of lactate turn point. As described herein, parameters of relating to the movement of the pelvis have shown correlation with a wider range of aspects of running performance than other kinematic parameters, for example relating to the centre of mass, and can be more accurately measured by a sensor incorporated or carried by a garment than kinematic parameters which have greater positional movement during physical activity.

Therefore, preferably, the one or more sensors may detect at least one parameter relating to movement of the pelvis of the user, for example relating to: the minimum forward pelvic velocity, such as the minimum forward pelvic velocity during each stride; the change in vertical position of the pelvis, such as the difference between the highest and lowest vertical position of the pelvis during each step, preferably normalised to height; the change in pelvis axial rotation, such as the axial rotation range of motion of the pelvis during each stride; the anterior angle of the pelvis, for example the minimum, maximum or mean anterior angle during each stride; the change in the anterior angle of the pelvis; or the vertical position of the pelvis, such as the lowest vertical position of the pelvis during ground contact. In some cases, the minimum forward pelvic velocity during each stride is measured for several strides, and then averaged to provide a representative average value of the pelvic velocity.

It has also been found that the analysis of the motion of the pelvis whilst an individual is running, in combination with the assessment of other selected kinematic variables, can further enhance the assessment of running technique. For example, it has been found that the analysis of a combination of the minimum velocity of the pelvis and the change in vertical position of the pelvis can explain a remarkable 17-37% of the variance in energy cost of running in a group of runners, including elite and recreational runners, at different speeds, and minimum velocity of the pelvis and the axial rotation of the pelvis can explain 15-25% of the variance in velocity of lactate turn point. Therefore, preferably the one or more sensors detect parameters relating to at least two aspects of the movement of the user, for example relating to at least two of: a parameter relating to movement of the pelvis of the user, a parameter relating to the ground contact of the user, a parameter relating to the stride pattern of the user and a parameter relating to the centre of mass of the user.

The one or more sensors may detect for example: a parameter relating to the velocity of the pelvis, preferably the minimum forward pelvic velocity, and a parameter relating to ground contact time; a parameter relating to the velocity of the pelvis, preferably the minimum forward pelvic velocity, and a parameter relating to the change in vertical position of the pelvis, such as the difference between the highest and lowest vertical position of the pelvis during each step, preferably normalised to height; a parameter relating to the velocity of the pelvis, preferably the minimum forward pelvic velocity and a parameter relating to the axial rotation of the pelvis, such as the axial rotation range of motion of the pelvis during each stride; a parameter relating to the velocity of the pelvis, preferably the minimum forward pelvic velocity, and a parameter relating to the change in velocity of the centre of mass of the user, such as the difference in anterior-posterior velocity of the centre of mass during stance between the minimum and maximum.

Furthermore, the one or sensors may also detect at least three parameters relating to the movement of the user, for example: a parameter relating to the velocity of the pelvis, preferably the minimum forward pelvic velocity, ground contact time, and the axial rotation of the pelvis; (ii) the velocity of the pelvis, preferably the minimum forward pelvic velocity, the axial rotation of the pelvis and the change in vertical position of the pelvis, preferably normalised to height. Such combinations of parameters have been found to be strongly correlated to the energy cost of running and velocity of lactate turn point.

It has also been found that the analysis of the parameters relating to ground contact, stride pattern and centre of mass of the user can have utility in the assessment of running technique, alone or in combination with assessment of the motion of the pelvis. Therefore, alternatively, or in addition, the one or more sensors may detect at least one parameter relating to the ground contact of the user, such as relating to: ground contact time (GCT); flight time (FLT); duty factor (DF); touch down to centre of mass (CM) distance, such as the anterior-posterior distance between the CM and toe at touch down, preferably normalised to height; take-off to centre of mass distance, such as the anterior-posterior distance between the CM and toe at take-off, preferably normalised to height; or ground contact distance, such as the sum of the anterior-posterior distance between the CM and toe at touch down and take-off, preferably normalised to height. Preferably, the one or more sensors may detect for example: a parameter relating to duty factor and a parameter relating to take-off to centre of mass distance, such as the anterior-posterior distance between the CM and toe at take-off; or a parameter relating to the ground contact distance, preferably normalised to height, and a parameter relating to lower spine angle, preferably relative to the angle during a standing stance.

Alternatively, or in addition, the one or more sensors may detect at least one parameter relating to the stride pattern of the user, such as relating to: stride rate (SR) or stride length, such as the anterior-posterior distance covered by the CM during a complete stride (e.g. right foot touch down to next right foot touch down), preferably normalised to height.

Alternatively, or in addition, the one or more sensors may detect at least one parameter relating to the centre of mass of the user, such as relating to: change in velocity of the centre of mass of the user, such as the difference in anterior-posterior velocity of the CM between the minimum and maximum, for example during stance, such as around take-off; or change in vertical position of the centre of mass of the user, such as the difference between the highest and lowest vertical position of the CM during each step (right foot touch down to left foot touch down).

It has also been found that the analysis of the parameters relating to the angles of spine, trunk and legs during running, and/or an analysis of the hip work done, can also have utility in the assessment of running technique, alone or in combination with assessment of the motion of the pelvis. Therefore, alternatively, or in addition, the one or more sensors may detect at least one parameter relating to lower spine angle, preferably relative to a measurement of the lower spine angle during a standing stance, such as the range of lower spine angle during each step, and/or hip work done, such as the positive work done at the hip joint during a flight/swing phase per unit body mass, and/or trunk angle, such as the axial rotation range of motion of the trunk during each stride.

Alternatively or in addition, the one or more sensors may detect at least one parameter relating to angles of the leg, such as foot strike angle, ankle angle at touchdown, shank angle at touchdown, changes in shank angle, such as the range of shank angle during ground contact, knee angle, such as the minimum knee angle during ground contact, or the hip angle at touchdown.

The system also includes a processing unit which is configured to receive information about the at least one parameter from the at least one sensor. The data received from the one or more sensors is compared to at least one aspect of a biomechanical model of the physical activity to determine if a feedback response is required.

The biomechanical model comprises information on a plurality of variables relating to the motion of a user undertaking the physical activity, such as a plurality of variables relating to different aspects of the motion of the user as detailed herein. Preferably, the biomechanical model comprises information relating to optimal ranges for each of the plurality of variables. Preferably the variables are selected and/or the optimal ranges generated by an analysis of the motion and performance of a plurality of individuals undertaking the physical activity. More preferably, the variables are selected and/or the optimal ranges are generated by an analysis of the motion of at least two groups of individuals with different performance levels, e.g. beginner and expert levels.

This biomechanical model may, for example, be based on an analysis of kinematic variables of importance to performance levels relating to the physical activity, for example in the case of running or activities involving running as is detailed herein.

The comparison with the biomechanical model may comprise analysis of one or more of: the velocity of the pelvis of the user, preferably the minimum forward pelvic velocity, such as the minimum forward pelvic velocity during each stride; the change in vertical position of the pelvis, such as the difference between the highest and lowest vertical position of the pelvis during each step, preferably normalised to height; the change in pelvis axial rotation, such as the axial rotation range of motion of the pelvis during each stride; or the anterior angle of the pelvis, for example the minimum, maximum or mean anterior angle; or the vertical position of the pelvis, such as the lowest vertical position of the pelvis during ground contact.

Alternatively, or in addition, the comparison with the biomechanical model may comprise analysis of one or more of: ground contact time; flight time; duty factor; touch down to centre of mass distance, such as the anterior-posterior distance between the CM and toe at touch down, preferably normalised to height; take-off to centre of mass distance, such as the anterior-posterior distance between the CM and toe at take-off, preferably normalised to height; or ground contact distance, such as the sum of the anterior-posterior distance between the CM and toe at touch down and take-off, preferably normalised to height.

Alternatively, or in addition, the comparison with the biomechanical model may comprise analysis of one or more of: stride rate or stride length, such as the anterior-posterior distance covered by the CM during a complete stride (e.g. right foot touch down to next right foot touch down), preferably normalised to height.

Alternatively, or in addition, the comparison with the biomechanical model may comprise analysis of one or more of: change in velocity of the centre of mass of the user, such as the difference in anterior-posterior velocity of the CM between the minimum and maximum, for example during stance, such as around take-off, or change in vertical position of the centre of mass of the user, such as the difference between the highest and lowest vertical position of the CM during each step (right foot touch down to left foot touch down).

Alternatively, or in addition, the comparison with the biomechanical model may comprise analysis of lower spine angle, preferably relative to lower spine angle during a standing stance, such as the range of lower spine angle during each step, and/or hip work done, such as the positive work done at the hip joint during a flight/swing phase per unit body mass, and/or trunk angle, such as the axial rotation range of motion of the trunk during each stride.

Alternatively or in addition, the comparison with the biomechanical model may comprise analysis of angles of the leg, such as foot strike angle, ankle angle at touchdown, shank angle at touchdown, changes in shank angle, such as the range of shank angle during ground contact, knee angle, such as the minimum knee angle during ground contact, or the hip angle at touchdown.

Preferably, the comparison with the biomechanical model may comprise analysis of at least two aspects of the movement of the user, for example relating to at least two of: the movement of the pelvis of the user, the ground contact of the user, the stride pattern of the user, and the centre of mass of the user.

The comparison with the biomechanical model may comprise an analysis of: the velocity of the pelvis, preferably the minimum forward pelvic velocity, and ground contact time; the velocity of the pelvis, preferably the minimum forward pelvic velocity, and the change in vertical position of the pelvis, such as the difference between the highest and lowest vertical position of the pelvis during each step, preferably normalised to height; the velocity of the pelvis, preferably the minimum forward pelvic velocity, and the axial rotation of the pelvis, such as the axial rotation range of motion of the pelvis during each stride; duty factor and take-off to centre of mass distance, such as the anterior-posterior distance between the CM and toe at take-off; the velocity of the pelvis, preferably the minimum forward pelvic velocity, and the change in velocity of the centre of mass of the user, such as the difference in anterior-posterior velocity of the CM between the minimum and maximum, for example during stance, such as around take-off; or ground contact distance, preferably normalised to height, and lower spine angle, preferably relative to the angle during a standing stance.

Alternatively or in addition, the comparison with the biomechanical model may comprise an analysis of (i) the velocity of the pelvis, preferably the minimum forward pelvic velocity, ground contact time, and the axial rotation of the pelvis; (ii) the velocity of the pelvis, preferably the minimum forward pelvic velocity, the axial rotation of the pelvis and the change in vertical position of the pelvis, preferably normalised to height.

The processing unit may, for example, compare the data received with an optimal range relating to an aspect of the biomechanical model and therefore determine whether a feedback response is required, for example in relation to the stride length of a runner, a determination as to whether the stride length is outside an optimal range.

The optimal range may be adjusted, for example, based on personal data entered by the user, for example relating to age, sex, height, weight etc. This range may also be adjusted based on contextual data, such as data collected over the period of the physical activity.

In the case where the data received being outside of an optimal range, the processing unit is configured to decide whether a feedback response is required. The processing unit may be configured to determine whether the provision of feedback relating to a single aspect of the biomechanical model would negatively impact on overall technique of the user, and therefore to determine whether to provide the feedback response. The processing unit may also be configured to determine if the system is currently providing a feedback response relating to an alternative aspect of the biomechanical model, and then decide whether to place the new feedback response in a queue or not to deliver the feedback response.

The system also comprises a means for providing a feedback response to the user, such as an audio speaker, visual display or apparatus for providing a mechanical or thermal stimulus. Preferably, this feedback is provided during the physical activity. This feedback may, for example, be provided by a means for providing a mechanical stimulus, for example by at least one actuator. Preferably the actuator is one or more of haptic actuator, thermal actuator, peltier tiles, transcutaneous electrical nerve stimulation (TENS) actuator, electro-active polymer or micro-piezo actuator, for example by at least one haptic actuator. This type of feedback response is not intrusive and enables the user to concentrate on the physical activity, without requiring reference to an audio or visual feedback mechanism.

The means for providing a mechanical stimulus, such as at least one haptic actuator, may be embedded in one or more garments forming part of the system. This enables the user to undertake the physical activity without the requirement to carry additional system components. The means for providing a mechanical stimulus, such as a haptic actuator, may be positioned to provide a feedback response at the location of the body at which a correction of technique is required, thereby enhancing the effectiveness of the feedback and helps the wearer to distinguish the action needed by them.

The feedback may also be by audio or visual means, for example through at least one speaker, headphones worn by the user and/or a visual display, etc. This feedback mechanism may be provided through a connection between the processing unit and a mobile electronic device, such as a smartphone, for example by means of a Bluetooth or other wireless connection. The system may be configurable to allow the user to customise the feedback response, for example, enabling the selection of the means of feedback response, or the priority of the delivery of feedback relating to different aspects of the biomechanical model.

The system may incorporate both mechanical and audio feedback mechanisms, for example through the combination of one or more haptic actuators and an audio and/or visual feedback mechanism.

The system may also be configured to enable to user to review analytical data relating to the run. For example, during or after a run data may be transferred to a software application, which may be configured to enable, for example, visualisation of post-run analytics, a comparison with historical data etc.

In a second aspect of the invention there is provided a garment for use in a system for monitoring the running technique of a user undertaking a physical activity, the garment comprising at least one sensor for the detection of at least one parameter relating to the motion of the user, an interface connector suitable for connecting to a processing unit, and wherein the or each sensor is connected to the interface connector by at least one data transmission path. Preferably, the at least one data transmission path is embedded with the garment, for example encapsulated on the inside of the garment.

The garment may be, for example, a garment worn on the lower half of the user's body, for example a pair of shorts, tights or leggings or a garment worn on the upper half of the user's body, such as a t-shirt or other running or training top or any other garment suitable for use during a physical activity. The garment is typically a base layer. Preferably the garment is close fitting to the body of the user. The garment incorporates at least one sensor which detects at least one parameter relating to the motion of the user. Such parameters may relate, for example to the speed, direction of movement, and/or acceleration of at least one part of the body of the user or to the relative speed, direction or movement and/or acceleration of two or more parts of the body. The sensor may, for example, be an accelerometer or gyroscope. The garment may include a combination of sensor types.

The garment may additionally comprise means for providing a feedback response to the user during the physical activity. This feedback may, for example, be provided by a means for providing a mechanical stimulus, for example by at least one actuator, where the actuator maybe one or more of haptic actuator, thermal actuator, peltier tiles, TENS actuator, electroactive polymer or micro-piezo actuator, for example by at least one haptic actuator. Preferably, the actuator is embedded in the garment.

The garment also includes an interface connector suitable for connecting to a processing unit. This connector enables the electrical and data connection between the processing unit and the at least one sensor. The connector may be arranged to enable a releasable connection between the garment and the processing unit, for example using snap connectors, such as magnetic snap connectors. This enables the processing unit to be exchanged between garments and removed before garment washing.

The interface connector provides a connection to the at least one sensor via at least one data transmission path embedded within the garment. This data transmission path enables transmission of data between the or each sensor and the processing unit. The data transmission paths may also provide an electrical connection between the system components. The data transmission paths may also connect the processing unit to the means for providing a feedback response to the user, such as an actuator.

In a third aspect of the invention there is provided a method for monitoring the running technique of an individual undertaking a physical activity, the method comprising the steps of:

(i) measuring at least one parameter relating to the motion of the individual;

(ii) comparing the or each parameter with at least one aspect of a biomechanical model of running to determine whether a feedback response is required;

(iii) providing the feedback response to the individual.

The method of the invention enables the individual to receive feedback on his or her running technique whilst undertaking a physical activity, such as distance running, football, or other sporting activities that involve the individual running, such as field hockey, rugby, lacrosse, orienteering for example. The distance running may be running or racing over distances such as 5k, 10k, marathons and half-marathons, or running/racing in events such as triathlon. The feedback provided enables the individual to enhance their technique, for example relating to their running form, such as knee positioning, hip positioning, stride length etc., or a combination of these factors. Maintaining and/or improving running technique during a physical activity can help to enhance performance, for example enhance endurance and/or speed and reduce the likelihood of injuries. Preferably, the feedback response is provided during the physical activity.

The method of the invention may involve the measurement of at least one parameter using at least one sensor incorporated within a garment worn by the individual, such as a garment as described herein. The garment could be, for example, a pair of shorts, a vest, a t-shirt, training top, leggings, or any other garment suitable for use during a physical activity. The garment is typically a base layer. Preferably the garment is close fitting to the body of the individual, for example close fitting to the torso, arms, legs etc. or any combination of these. The method may use sensors incorporated within a combination of garments, for example a combination of a garment worn on the lower half of the individual's body and a garment worn on the upper half, such as a pair of shorts or leggings and a t-shirt or training top. This enables sensors to be placed in positions to monitor motion in the both the upper body, such as the arms, and lower body, such as the legs.

The method may alternatively, or in addition, utilise at least one sensor attached to the body of the individual, attached to a garment worn by the individual, attached to or incorporated in a shoe or shoes worn by the individual, incorporated in a device carried by or attached to the individual, such as a portable electronic device or watch, or may utilise other methodology such as video recording and analysis. The method may also be used to provide feedback on the running style of an individual on the treadmill.

The method involves the measurement of at least one parameter relating to the motion of the individual. Such parameters may relate, for example, to the speed, direction of movement, and/or acceleration of at least one part of the body of the individual or to the relative speed, direction or movement and/or acceleration of two or more parts of the body, or other kinematic data. The measurement may involve the use of a sensor, for example, an accelerometer or a gyroscope. The method may use a combination of sensor types, for example a combination of at least one accelerometer and at least one gyroscope.

As described herein, it has been found that the analysis of the motion of the pelvis whilst an individual is running, alone or in combination with other kinematic variables, can have high utility in the assessment of running technique. Therefore, the method may comprise the measurement of at least one parameter relating to movement of the pelvis of the individual, for example relating to: the minimum forward pelvic velocity, such as the minimum forward pelvic velocity during each stride; the change in vertical position of the pelvis, such as the difference between the highest and lowest vertical position of the pelvis during each step, preferably normalised to height; the change in pelvis axial rotation, such as the axial rotation range of motion of the pelvis during each stride; the anterior angle of the pelvis, for example the minimum, maximum or mean anterior angle; or the vertical position of the pelvis, such as the lowest vertical position of the pelvis during ground contact.

Alternatively, or in addition, the method may comprise the measurement of at least one parameter relating to: the ground contact of the individual, such as relating to ground contact time (GCT); flight time (FLT); duty factor (DF); touch down to centre of mass distance (CM), such as the anterior-posterior distance between the CM and toe at touch down, preferably normalised to height; take-off to centre of mass distance, such as the anterior-posterior distance between the CM and toe at take-off, preferably normalised to height; or ground contact distance, such as the sum of the anterior-posterior distance between the CM and toe at touch down and take-off, preferably normalised to height.

Alternatively, or in addition, the method may comprise the measurement of at least one parameter relating to: the stride pattern of the individual, such as relating to stride rate (SR); or stride length, such as the anterior-posterior distance covered by the CM during a complete stride (e.g. right foot touch down to next right foot touch down), preferably normalised to height.

Alternatively, or in addition, the method may comprise the measurement of at least one parameter relating to: the centre of mass of the individual, such as relating to change in velocity of the centre of mass of the individual, such as the difference in anterior-posterior velocity of the CM between the minimum and maximum, for example during stance, such as around take-off; or change in vertical position of the centre of mass of the individual, such as the difference between the highest and lowest vertical position of the CM during each step (right foot touch down to left foot touch down).

Alternatively, or in addition, the method may comprise the measurement of at least one parameter relating to lower spine angle, preferably relative to lower spine angle during a standing stance, and/or hip work done, such as the positive work done at the hip joint during a flight/swing phase per unit body mass, and/or trunk angle, such as the axial rotation range of motion of the trunk during each stride.

Alternatively or in addition, the method may comprise the measurement of at least one parameter relating to angles of the leg, such as foot strike angle, ankle angle at touchdown, shank angle at touchdown, changes in shank angle, such as the range of shank angle during ground contact, knee angle, such as the minimum knee angle during ground contact, or the hip angle at touchdown.

Preferably, the method comprises measurement of parameters relating to at least two aspects of the movement of the individual, for example relating to at least two of: a parameter relating to movement of the pelvis of the individual, a parameter relating to the ground contact of the individual, a parameter relating to the stride pattern of the individual and a parameter relating to the centre of mass of the individual.

The method may comprise the measurement of, for example: a parameter relating to the velocity of the pelvis, preferably the minimum forward pelvic velocity, and a parameter relating to ground contact time; a parameter relating to the velocity of the pelvis, preferably the minimum forward pelvic velocity, and a parameter relating to the change in vertical position of the pelvis, such as the difference between the highest and lowest vertical position of the pelvis during each step, preferably normalised to height; a parameter relating to the velocity of the pelvis, preferably the minimum forward pelvic velocity and a parameter relating to the axial rotation of the pelvis, such as the axial rotation range of motion of the pelvis during each stride; a parameter relating to duty factor and a parameter relating to take-off to centre of mass distance, such as the anterior-posterior distance between the CM and toe at take-off; a parameter relating to the velocity of the pelvis, preferably the minimum forward pelvic velocity, and a parameter relating to the change in velocity of the centre of mass of the individual. such as the difference in anterior-posterior velocity of the CM during stance between the minimum and maximum; a parameter relating to the ground contact distance, preferably normalised to height, and a parameter relating to lower spine angle, preferably relative to the angle during a standing stance.

Alternatively or in addition, the method may comprise the measurement of, for example, a parameter relating to the velocity of the pelvis, preferably the minimum forward pelvic velocity, ground contact time, and the axial rotation of the pelvis; or (ii) the velocity of the pelvis, preferably the minimum forward pelvic velocity, the axial rotation of the pelvis and the change in vertical position of the pelvis, preferably normalised to height.

The or each measured parameter is compared with at least one aspect of a biomechanical model of running to determine if a feedback response is required. The biomechanical model comprises information on a plurality of variables relating to the motion of an individual whilst running, such as a plurality of variables relating to different aspects of the motion of the individual whilst running as detailed herein. Preferably, the biomechanical model comprises information relating to optimal ranges for each of the plurality of variables. Preferably the variables are selected and/or the optimal ranges generated by an analysis of the motion and performance of a plurality of individuals undertaking the physical activity. More preferably, the variables are selected and/or the optimal ranges are generated by an analysis of the motion of at least two groups of individuals with different performance levels, e.g. beginner and expert levels, or by assessing the relationship between a kinematic variable with running performance and economy.

This biomechanical model may, for example, be based an analysis of kinematic variables of importance to running performance levels, for example as is detailed herein.

The comparison with the biomechanical model may comprise analysis of one or more of the velocity of the pelvis of the individual, preferably the minimum forward pelvic velocity, such as the minimum forward pelvic velocity during each stride; the change in vertical position of the pelvis, such as the difference between the highest and lowest vertical position of the pelvis during each step, preferably normalised to height; the change in pelvis axial rotation, such as the axial rotation range of motion of the pelvis during each stride; or the anterior angle of the pelvis, for example the minimum, maximum or mean anterior angle; or the vertical position of the pelvis, such as the lowest vertical position of the pelvis during ground contact

Alternatively, or in addition, the comparison with the biomechanical model may comprise analysis of one or more of ground contact time; flight time; duty factor; touch down to centre of mass distance, such as the anterior-posterior distance between the CM and toe at touch down, preferably normalised to height; take-off to centre of mass distance, such as the anterior-posterior distance between the CM and toe at take-off, preferably normalised to height; or ground contact distance, such as the sum of the anterior-posterior distance between the CM and toe at touch down and take-off, preferably normalised to height.

Alternatively, or in addition, the comparison with the biomechanical model may comprise analysis of stride rate, or stride length, such as the anterior-posterior distance covered by the CM during a complete stride (e.g. right foot touch down to next right foot touch down), preferably normalised to height. Alternatively, or in addition, the comparison with the biomechanical model may comprise analysis of change in velocity of the centre of mass of the individual, such as the difference in anterior-posterior velocity of the CM between the minimum and maximum, for example during stance, such as around take-off; or change in vertical position of the centre of mass of the individual, such as the difference between the highest and lowest vertical position of the CM during each step (right foot touch down to left foot touch down).

Alternatively, or in addition, the comparison with the biomechanical model may comprise analysis of lower spine angle, preferably relative to an angle during a standing stance, such as the range of lower spine angle during each step, and/or hip work done, such as the positive work done at the hip joint during a flight/swing phase per unit body mass and/or trunk angle, such as the axial rotation range of motion of the trunk during each stride

Alternatively or in addition, the comparison with the biomechanical model may comprise analysis of angles of the leg, such as foot strike angle, ankle angle at touchdown, shank angle at touchdown, changes in shank angle, such as the range of shank angle during ground contact, knee angle, such as the minimum knee angle during ground contact, or the hip angle at touchdown.

Preferably, the comparison with the biomechanical model may comprise analysis of at least two aspects of the movement of the individual, for example relating to at least two of: the movement of the pelvis of the individual, to the ground contact of the individual, the stride pattern of the individual and the centre of mass of the individual.

The comparison with the biomechanical model may comprise an analysis of: the velocity of the pelvis, preferably the minimum forward pelvic velocity, and ground contact time; the velocity of the pelvis, preferably the minimum forward pelvic velocity, and the change in vertical position of the pelvis, such as the difference between the highest and lowest vertical position of the pelvis during each step, preferably normalised to height; the velocity of the pelvis, preferably the minimum forward pelvic velocity, and the axial rotation of the pelvis, such as the axial rotation range of motion of the pelvis during each stride; duty factor and take-off to centre of mass distance, such as the anterior-posterior distance between the CM and toe at take-off; the velocity of the pelvis, preferably the minimum forward pelvic velocity, and the change in velocity of the centre of mass of the individual, such as the difference in anterior-posterior velocity of the CM during stance between the minimum and maximum; or ground contact distance, preferably normalised to height, and lower spine angle, preferably relative to the angle during a standing stance.

Alternatively or in addition, the comparison with the biomechanical model may comprise an analysis of (i) the velocity of the pelvis, preferably the minimum forward pelvic velocity, ground contact time, and the axial rotation of the pelvis; (ii) the velocity of the pelvis, preferably the minimum forward pelvic velocity, the axial rotation of the pelvis and the change in vertical position of the pelvis, preferably normalised to height.

The comparison with the biomechanical model may involve a comparison with an optimal range relating to an aspect of the biomechanical model and this therefore enables the determination as to whether a feedback response is required, for example in relation to the stride length of a runner, a determination as to whether the stride length is outside an optimal range.

The optimal range may be adjusted, for example, based on personal data entered by the individual, for example relating to height or weight. This range may also be adjusted based on contextual data, such as data collected over the period of the physical activity.

The method may involve the step of determining whether the provision of feedback relating to a single aspect of the biomechanical model would negatively impact on the overall technique of the individual, and therefore determining whether to provide the feedback response. The method may also involve the step of determining whether the system is currently providing a feedback response relating to an alternative aspect of the biomechanical model, and then deciding whether to place the new feedback response in a queue or not to deliver the feedback response.

The method comprises the step of providing a feedback response to the individual. Preferably, this feedback is provided during the physical activity. This feedback may, for example, be provided by an audio speaker, visual display or apparatus for providing a mechanical or thermal stimulus, such as a means for providing a mechanical stimulus, for example by at least one actuator. Preferably the actuator is one or more of haptic actuator, thermal actuator, peltier tiles, transcutaneous electrical nerve stimulation (TENS) actuator, electro-active polymer or micro-piezo actuator, for example by at least one haptic actuator. Mechanical stimulus as a feedback response is not intrusive and enables the individual to concentrate on the physical activity, without requiring reference to an audio or visual feedback mechanism.

The means for providing a mechanical stimulus, such as at least one haptic actuator, may be embedded in one or more garments worn by the individual. This enables the individual to undertake the physical activity without the requirement to carry additional items. The means for providing a mechanical stimulus, such as a haptic actuator, may be positioned to provide a feedback response at the location of the body at which a correction of technique is required, thereby enhancing the effectiveness of the feedback and helps the wearer to distinguish the action needed by them.

The feedback may also be by an audio signal or visual display, for example through at least one speaker, headphones worn by the individual, and/or a visual display etc. This feedback mechanism may be provided through a connection between the processing unit and a mobile electronic device, such as a smartphone, for example by means of a Bluetooth or other wireless connection.

The feedback means may incorporate both mechanical and audio feedback mechanisms, for example through the combination of one or more haptic actuators and an audio and/or visual feedback mechanism.

The present invention will now be described, by way of example only, with reference to the accompanying drawings, in which:

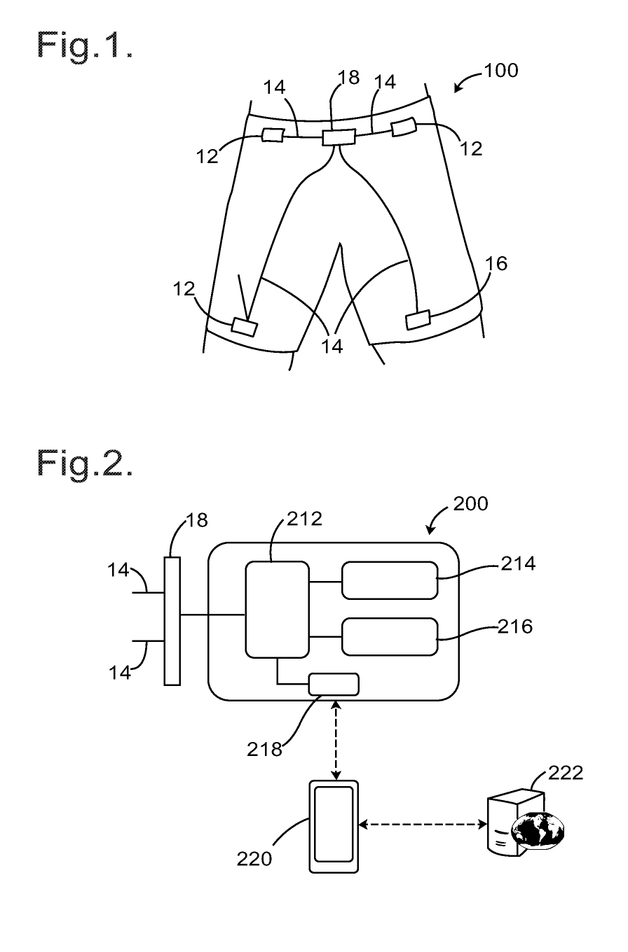

FIG. 1 shows a schematic representation of an embodiment of a garment according to the invention.

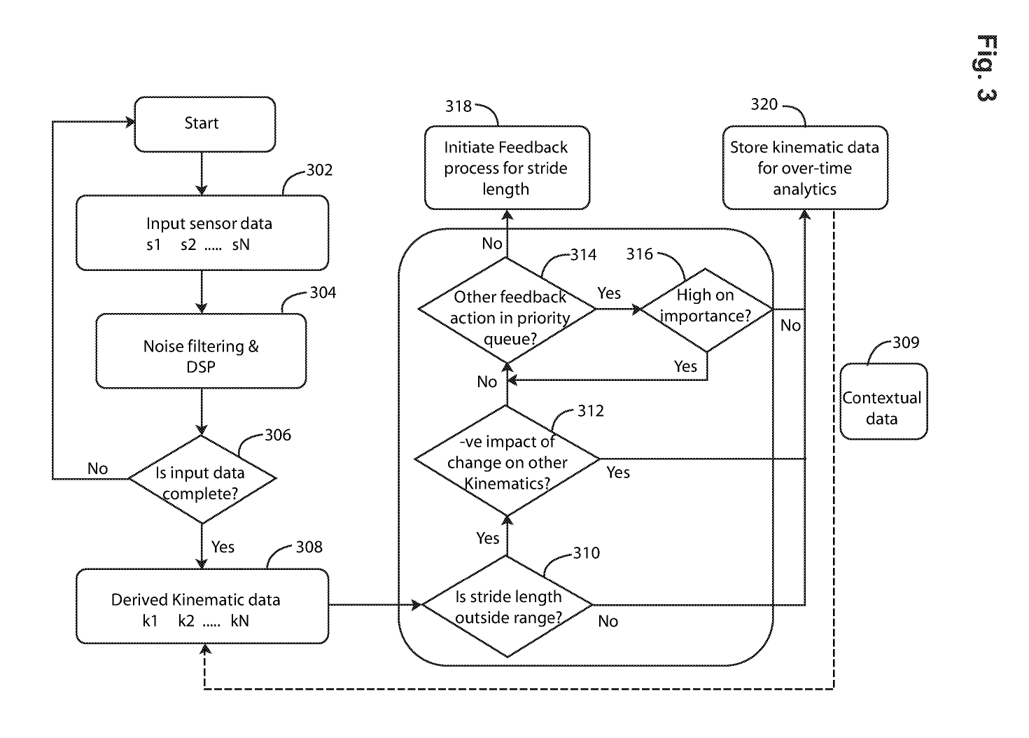

FIG. 2 shows a schematic representation of a processing unit for use in a system according to the invention.

FIG. 3 shows an embodiment of a logic flow for determining whether a feedback response is required.

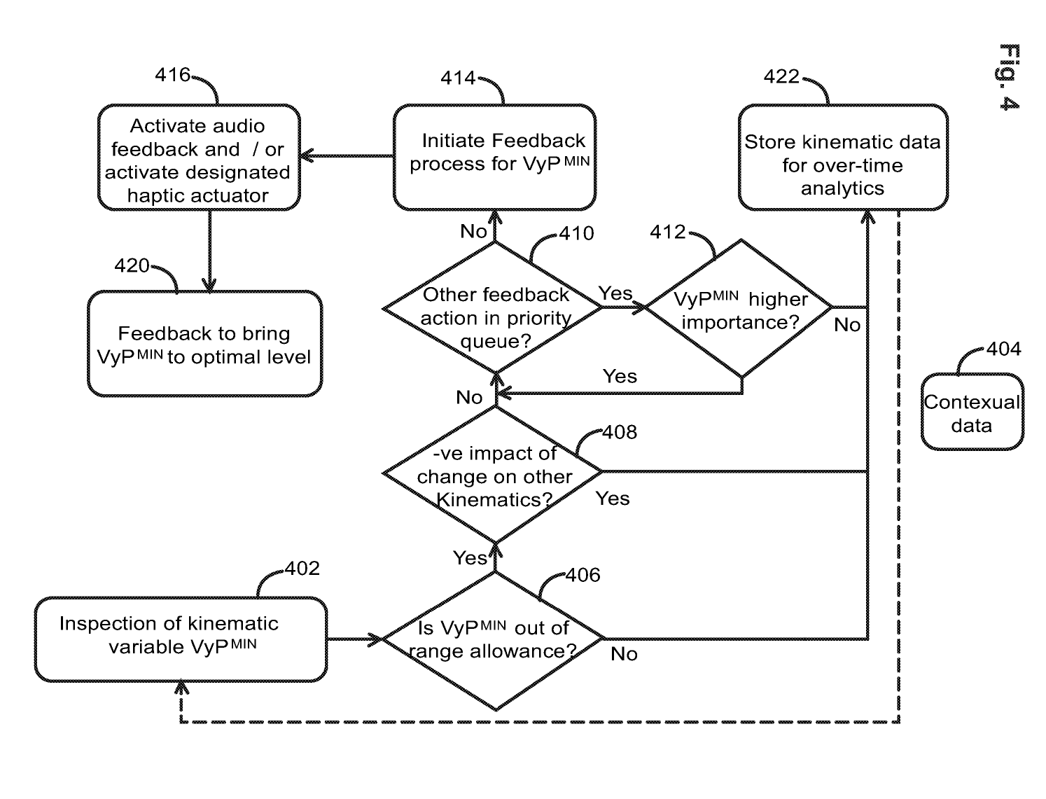

FIG. 4 shows an embodiment of a logic flow for determining whether a feedback response is required using the minimum pelvic velocity.

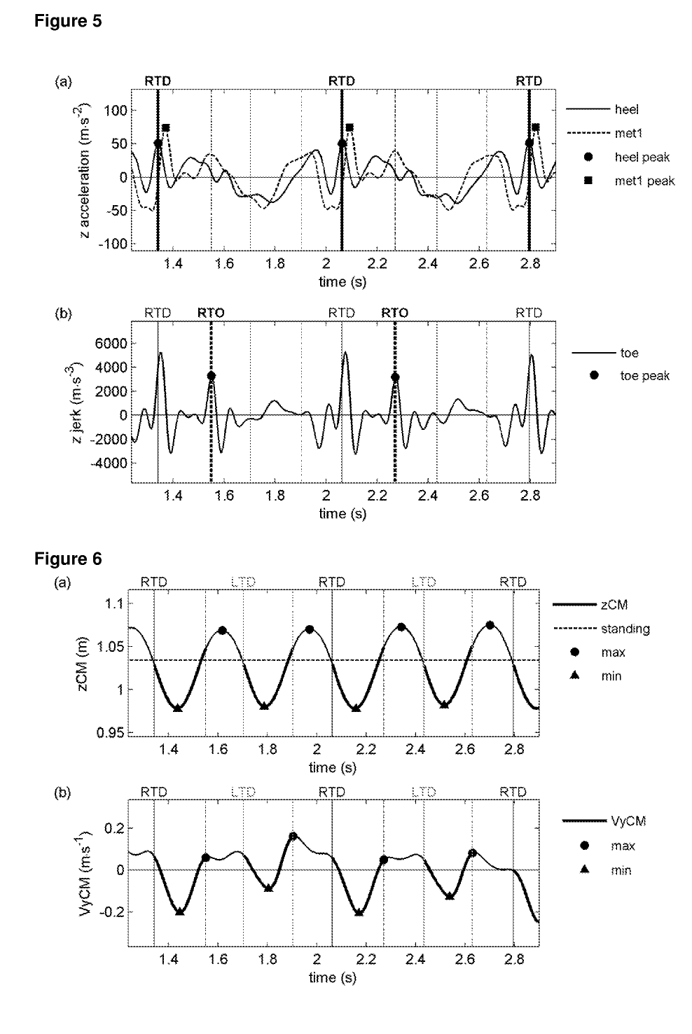

FIG. 5 shows an example of touchdown and take-off identification.

FIG. 6 shows an example of methodology for the measurement of centre of mass vertical movement and anterior-posterior velocity.

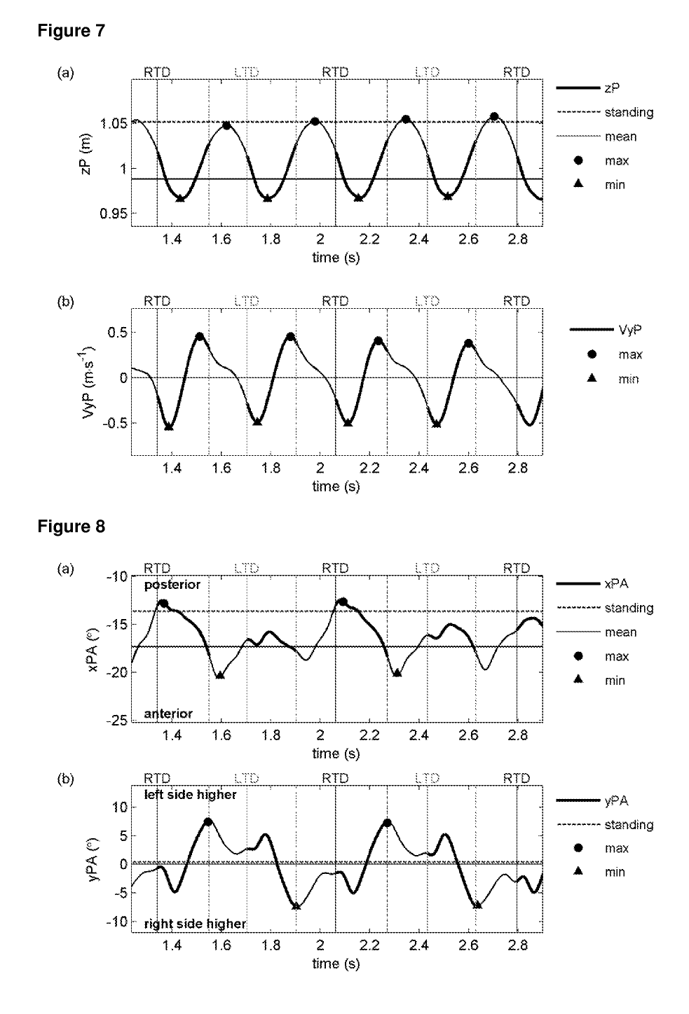

FIG. 7 shows an example of methodology for the measurement of pelvis vertical position and anterior-posterior velocity of the pelvis.

FIG. 8 shows an example of methodology for the measurement of pelvis rotation angles.

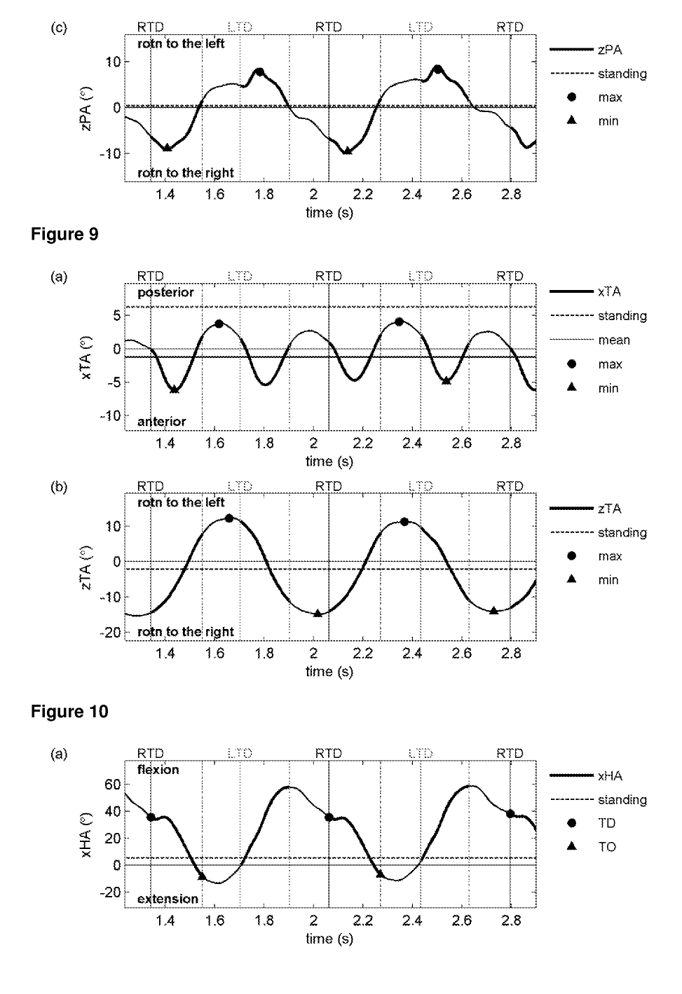

FIG. 9 shows an example of methodology for the measurement of trunk rotations.

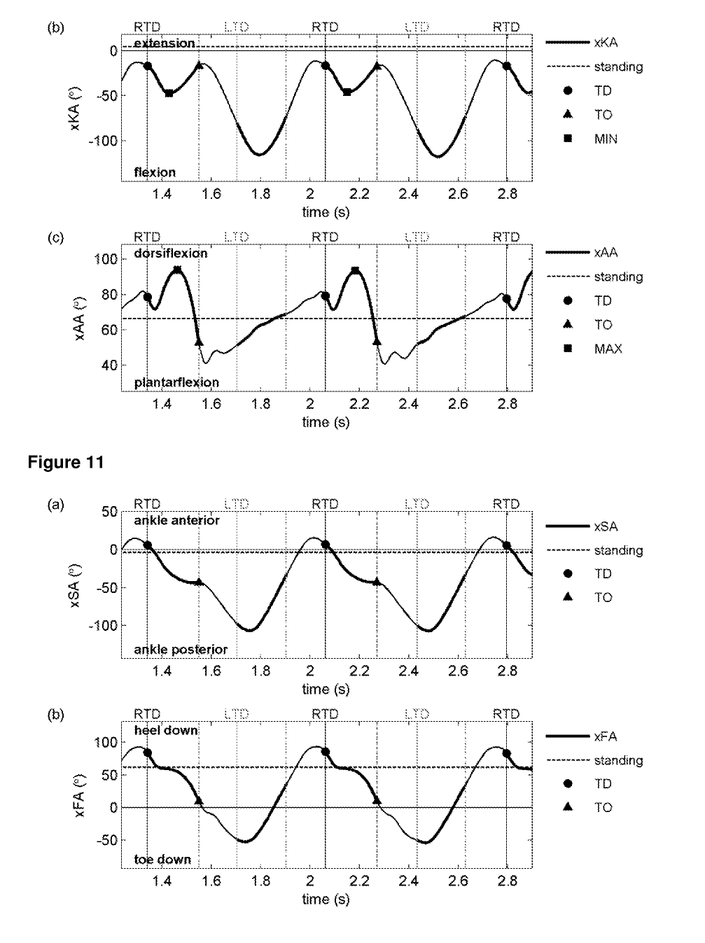

FIG. 10 shows an example of methodology for the measurement of lower limb flexion-extension angles.

FIG. 11 shows an example of methodology for the measurement of foot and shank angles.

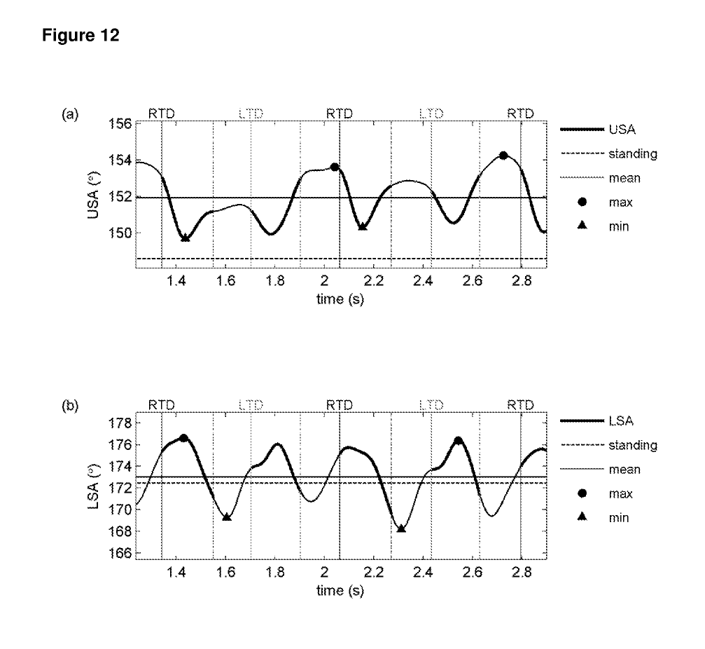

FIG. 12 shows an example of methodology for the measurement of upper and lower sagittal plane spine angles.

### DETAILED DESCRIPTION OF THE INVENTION

### Abbreviations

GCT Ground contact time. The time the foot is in contact with the ground, i.e. time from the instant of touchdown to toe-off.
FLT Flight time. The time that neither foot is on contact with the ground, i.e. time from the instant of toe-off for one foot to touch down for the other foot.
DF Duty factor. Proportion of the stride that the foot is in contact with the ground
SR Stride rate. Number of strides per unit time.
CM Centre of mass
TD Touchdown. Point of initial contact between the foot and the floor
TO Toe off. Point of final contact between the surface and the toe, before the initiation of the flight phase
GC Ground contact phase. Phase during which the foot of the measured leg is on contact with the ground.
SW Swing phase. Phase during which the foot of the measured leg is not in contact with the ground.
Ec Energy cost of running
\dot{V}O₂max Maximal oxygen uptake

FIG. 1 shows a garment 100 suitable for running which includes sensors 12. The sensors 12 detect one or more parameters relating to the motion of the user which are important to running form and technique. The sensors 12 are connected to an interface connector 18 by data transmission paths 14 which are also used to provide power to the sensors 12. The data transmission paths 14 are encapsulated on the inside of the garment 100. The garment 100 also includes a haptic actuator 16 which provides a feedback response to the user regarding form and technique whilst the user is running. The haptic actuator 16 is connected to the interface connector 18 by a data transmission path 14.

The interface connector 18 enables the releasable connection of processing unit 200 (FIG. 2) to the garment 100. The interface connector 18 enables data and power transfer between the processing unit 200 and the sensors 12 (not shown in FIG. 2) and the haptic actuator 16 (not shown in FIG. 2) via the data transmission paths 14. In use, the processing unit activates the sensors 12 and the haptic actuator in the garment. The processing unit 200 comprises a processor 212, a memory module 214, a battery 216 and a wireless enabling unit 218. The processor 212 receives data from the sensors 12 and compares the parameters with at least one aspect of a biomechanical model of running in order to monitor current running form and technique. The processor 212 determines whether a feedback response is required.

The memory module 214 is used, for example, to store relevant data points which can be used in a post-run analysis of form and technique and/or to provide contextual data to adjust the analysis carried out by the processing unit 200 during the run. The memory module 214 can store data from multiple activity instances until, for example, they are transmitted to an external device.

The processing unit 200 may be connected to a mobile phone or other portable electronic device 220 via wireless enabling unit 218, which may for example set up a Bluetooth connection. The portable electronic device 220 is provided with a software application which can process data accumulated during the physical activity and provide post-run and historical analysis, tips and information on the users form and technique. The software application may use cloud based storage 222 as a back-up repository for this accumulated data as well as a platform to share this data with other applications, devices or human coaches as selected/configurable by the user. The wireless connection between the portable electronic device 220 and the processing unit 200 may also be used to update software on the processing unit 200 (including but not limited to the biomechanical model embedded in the processing unit 200).

This feedback response may be delivered via the haptic actuator 16 in the garment or, if the processing unit 200 is connected to a portable electronic device 220 whilst the user is running, then the software application may deliver audio and/or visual feedback, or feedback though, for example, vibration of the portable electronic device 220.

In use, the user will download a software application to the portable electronic device 220 and will connect the processing unit 200 to the software application (for example via Bluetooth) and enter their personal variables to customize the feedback response to their profile.

The user will attach the processing unit 200 to the garment 100 prior to starting the run and can optionally connect the processing unit 200 to the software application if the user plans to take a portable electronic device 220 on the run.

The user will run as usual. The system will monitor the form and technique of the user and provide improvement feedback via the haptic actuator 16 (the user can customize the frequency and type of the feedback via the software application or turn off feedback from the processing unit as their preference). Optionally, the user can receive feedback in audio/visual form from the software application if it's connected to the processing unit 200 while running. This feedback may, for example, be delivered through headphones.

The user may elect to adjust their running form or technique according to the feedback given. The system will learn some of the intrinsic and unique features of the user in order to adapt future feedback. The user will finish the run.

The user can connect the processing unit 200 to the software application (if not done before) to transfer the run data to the application. The user will review post-run analytics and historical data on the software application. The software application will back-up to the cloud 222. If the processing unit is not connected to the software application after a particular run, it will retain the data until the connection is made.

The biomechanical model can reside in the firmware of the processing unit and/or on the code of the software application. Any changes to biomechanical model may be updated to the user by updating the software application and pushing the firmware update wirelessly to the processing unit 200.

### Example of the System in Use

SCENARIO: A recreational runner is on a training run with an embodiment of the system of the invention. The system comprises of a pair of shorts coupled with the detachable processing unit 200. The runner will have previously entered their basic bio-physical information (such as age, weight, height) to the system via the software application and the processing unit 200 is attached to the garment via the interface connector 18. The system in this embodiment has seven sensors 12 and a three haptic actuators 16 integrated into the shorts. The sensors 12 are placed to detect the kinematic parameters required to monitor running form and technique and the actuators 16 are placed to deliver discernible feedback in key areas of the body that helps the wearer to distinguish the action needed by them.

The shorts are constructed as follows:
Base fabric: Polyester/Spandex or Nylon/Polyester/Spandex composite, Synthetic fiber
Construction: Fitted to the body, close fit, light compression
Conductive mechanism: Twisted stretchable conductive yarn for sensors, pattern laid stainless steel yarn for actuators (to account for different load delivery)
Sensor encapsulation: moulded silicone
Actuator encapsulation: polyurethane tape+textile
Conductive path encapsulation: polyurethane tape+textile
Apparel electronic interface: Magnetic snaps in flexible molded thermoplastic polyurethane

FIG. 3 shows a logic flow used by the system to monitor stride length. The system would recognize when the runner starts the training run through accelerometry data from the sensors 12 and start polling the sensors 12 and analyzing the data. There are allowances built in to allow the relatively chaotic data from the ‘warm-up’ and ‘cool-down’ periods to be filtered out so that any unnecessary or premature feedback is not given to the runner. Once the system detects that the runner is in a natural running motion, the sensor data is imputted, step 302, and the systematic interpretation of sensor data starts.

The sensor data will be subject to a noise filtering algorithm at step 304, and subjected to digital signal processing (DSP). The sensors are polled at a set frequency, and the processed kinematic data will be normalized before the system decides that the input data is complete, step 306.

The derived kinematic data 308 will be inspected for parameters and optimal ranges determined by the biomechanical model. The allowances are stored in the firmware as a dynamic variable which will be fine-tuned or personalized to the individual runner over time through the analysis of trends and the runners' reaction to the feedback given by the system.

The real-time kinematic data will be inspected in conjunction with over-time data (the trend) to provide context 309. Upon inspection of data, the system will decide if the kinematic parameter is within optimal ranges or not in the current context—for example in

FIG. 3 that the stride length is outside of a range allowance 310.

In the case of the kinematic parameter being out of an optimal range, the system will further decide if any feedback on the particular kinematic would be detrimental to the performance of the runner in the current context of all (holistic) kinematic data captured, for example will changing stride length affect other current kinematics negatively, step 312. The feedback component of the biomechanical model will dictate how to gauge the impact of changing kinematics on other biomechanical aspects.

The system will further check at step 314 if there is additional feedback currently in progress, or queued before delivering feedback on the out of range kinematic, as concurrent or sequential feedback may be less effective. Depending on the importance the system attaches to the kinematic variable and the feedback at step 316, the data will either be stored for future contextual analysis or put in a queue.

When a kinematic variable is due for feedback, the actuator management system will take over and deliver the appropriate haptic feedback at step 318; at the correct location for the kinematic variable, in the correct duration, intensity and pattern (i.e. a simple ‘taptic language’).

The runner (wearer of the system) can elect to react or not react to the haptic feedback given, but given the runner's objective and the intuitive nature of the feedback it is very much likely that they will adjust and adapt to the given feedback

The system will recursively continue to capture, and inspect kinematic data and provide relevant feedback, until the running activity ceases.

The runner (wearer of the system) will be able to adjust the amount and type of feedback (i.e. which kinematic variables will take priority) through the software application.

The stored data 320 after each ‘run’ (as recognized automatically by the system itself) can be transmitted to the software application for further analysis, graphical representation and the formulation of a score for each of the kinematic categories—indicating how close the particular ‘run’ has fared against the biomechanical standard. An overall ‘run score’ would also be calculated from the weighted average of the scores for kinematic categories.

As an alternative, in situations in which the runner carries the portable electronic device 220 with them on the run, the system will transfer the particular kinematic information (via Bluetooth wireless transmission for example) to the software application where it will be indicated as a message/indicator on the screen for a predetermined duration as well as a vibration from the device's vibrator (if enabled).

FIG. 4 shows an example of a logic flow for use in a method of monitoring the running technique of an individual. The exemplified method involves an analysis at step 402 of the minimum forward pelvic velocity (VyP^{MIN}) of the individual. This data may be gathered, for example, from a motion sensor embedded in a garment worn by the individual, such as a pair of running shorts. The analysis at step 402 may involve the average VyP^{MIN} over a number of steps and/or the trend in VyP^{MIN} over a number of steps. The VyP^{MIN} may be inspected in conjunction with over-time data (the trend) to provide context 404. The VyP^{MIN} values are compared at step 406 with an optimal range for the current running pace of the individual. This optimal range may be identified by an analysis of the VyP^{MIN} of a plurality of runners at a given pace together with an analysis of metrics relating to their running efficiency.

If the VyP^{MIN} is determined to be less than optimal for the current pace, the method involves at step 408 an analysis of whether changing VyP^{MIN} would affect other higher priority kinematics negatively and then at step 410 and step 412, whether other higher priority feedback action is already queued. If it is determined that no higher priority feedback is queued then the method involves at step 414 the initiation of a feedback process to the individual for VyP^{MIN}. This may involve at step 416 the activation of audio feedback and/or a designated haptic actuator, for example an actuator embedded in a piece of clothing worn by the individual. This feedback response may be a simple alert, for example an audible or mechanical alert, that the VyP^{MIN} is outside an optimal range, in response to which the individual may alter an element of their running style with the aim of regaining the optimal parameter range. The feedback response may alternatively, or in addition, include an instruction to the user to modify an element of their running style, for example by a mechanical stimulus at a specific point of the individual's body or an audible command, such as “increase your cadence” or “tuck in your pelvis”. VyP^{MIN} gathered whilst the individual is running is stored at step 422 for use in over-time analytics.

As described, verbal prompts may be provided to assist the user in improvements to their technique. For example, suitable verbal prompts for ground contact variables (such as GCT, GCD, DF, FSA, TD-CM, TO-CM, AA_{TD}, SA_{TD}, HA_{TD}, etc.) may include “run tall”, “straighten back”, “look ahead not down”, with the aim of getting the user to lift the hips, and therefore have a foot strike closer to their centre of mass, and result in the user running with a flatter foot strike. Such prompts may also be suitable for variables including xTA, VyP^{min}, KA_{MIN}, ΔVyCM. Suitable prompts relating to trunk angles, include “drive the arm back”, “drive the elbows back”, “use the arms”, “tighten your core” and “tense your abs”.

### Nomenclature Used for Biomechanical Variables

### Temporal-Spatial Parameters

| | | | |
|---|---|---|---|
| 1 | Ground contact | GCT | Time the foot is in contact with the ground, i.e. time from the |
| | time | | instant of touchdown (TD) to toe-off (TO). |
| 2 | Flight time | FLT | Time that neither foot is in contact with the ground, i.e. time from |
| | | | the instant of TO for one foot to TD for the other foot. |
| 3 | Ground contact | GCD | Sum of the anterior-posterior distance between the centre of |
| | distance (H) | | mass (CM) and toe at TD and TO. |
| 4 | Touchdown to | TD − | Anterior-posterior distance between the CM and toe at TD |
| | centre of mass | CM | |
| | distance (H) | | |
| 5 | Take-off to centre | TO − | Anterior-posterior distance between the CM and toe at TO |
| | of mass distance | CM | |
| | (H) | | |
| 6 | Stride length (H₎ | SL | Anterior-posterior distance covered by the CM during a |
| | | | complete stride (right foot TD to next right foot TD). |
| 7 | Stride rate | SR | Number of strides per unit time. |
| 8 | Duty factor | DF | Proportion of the stride that the foot is in contact with the |
| | | | ground. |
| 9 | Step width (H) | SW | Medial-lateral distance between the CM and mid-point of the |
| | | | heel and hallux markers at TD. |

### Lower Limb Joint and Segment Angles

| | | | |
|---|---|---|---|
| 1 | Footstrike angle | FSA/ | Flexion-extension angle of the foot (angle of the foot in the |
| | | xFA | sagittal plane) with reference to the lab co-ordinate system |
| | | | (LCS) at TD. Measured relative to the quiet standing trial. |
| | | | Positive represents heelstrike. |
| 2 | Hip angle | HA/ | Flexion-extension angle between the pelvis and thigh segments |
| | | xHA | (e.g. obtained at TD and TO). Measured relative to the quiet |
| | | | standing trial. Positive represents flexion. |
| 3 | Knee angle | KA/ | Flexion-extension angle between the thigh and shank segments |
| | | xKA | (e.g. obtained at TD, TO and the maximum flexion during |
| | | | stance). Measured relative to the quiet standing trial. Negative |
| | | | represents flexion. |
| 4 | Knee abduction | KAA/ | (Maximum) abduction-adduction angle between the thigh and |
| | angle | yKA | shank segments. Measured relative to the quiet standing trial. |
| | | | Positive represents abduction. |
| 5 | Ankle angle | AA/ | Dorsi-plantar flexion angle between the shank and foot |
| | | xAA | segments (e.g. obtained at TD, TO and maximum dorsi-flexion |
| | | | during stance). Measured relative to the quiet standing trial. |
| | | | Positive represents dorsiflexion. |
| 6 | Shank angle | SA/ | Flexion-extension angle of the shank (angle of the shank in the |
| | | xSA | sagittal plane) with reference to the lab-co-ordinate system |
| | | | (LCS) (e.g. obtained at TD and TO and the ground contact |
| | | | range, ΔSA). Measured relative to the quiet standing trial. |
| | | | Positive represents the distal end of the shank being in a more |
| | | | anterior (forward) position. |

### Movement of the Centre of Mass

| | | | |
|---|---|---|---|
| 1 | Vertical position | zCM | Vertical position of the CM, measured relative to the quiet |
| | of the centre of | | standing trial |
| | mass | | |
| 2 | Change in | ΔzCM | Difference between the highest and lowest vertical position of |
| | vertical position | | the CM during each step (right foot TD to left foot TD). |
| | of the centre of | | |
| | mass(H) | | |
| 3 | Anterior velocity | VyCM | Anterior-posterior velocity of the CM |
| | of the centre of | | |
| | mass | | |
| 4 | Change in | ΔVyCM | Difference in anterior-posterior velocity of the CM during stance |
| | velocity of the | | between the minimum and maximum. |
| | whole body CoM | | |

### Movement and Orientation of the Pelvis

| | | | |
|---|---|---|---|
| 1 | Change in | ΔzP | Difference between the highest (^{MAX}) and lowest (^{MIN}) vertical |
| | vertical position | | position of the pelvis during each step (right foot TD to left foot |
| | of the pelvis (H) | | TD). |
| 2 | Change in | ΔVyP | Difference between the minimum and maximum around TO in |
| | velocity of the | | anterior-posterior velocity of the pelvis during stance. |
| | pelvis | | |
| 3 | Velocity of the | VyP | Anterior-posterior velocity of the pelvis |
| | pelvis | | |
| 4 | Vertical position | zP | Vertical position of the pelvis. Measured relative to the quiet |
| | of the pelvis (H) | | standing trial. |
| 5 | Anterior lean of | xPA | Flexion-extension angle of the pelvis. Measured relative to the |
| | the pelvis | | quiet standing trial value. Negative represents flexion (anterior |
| | | | or forward lean). |
| 6 | Change in pelvis | ΔxPA | Tilt (flexion-extension) range of motion of the pelvis during each |
| | tilt | | stride. |
| 7 | Change in pelvis | ΔyPA | Obliquity (side-to-side) range of motion of the pelvis during each |
| | obliquity | | stride. |
| 8 | Change in pelvis | ΔzPA | Axial rotation range of motion of the pelvis during each stride |
| | axial rotation | | (zPA, axial rotation of the pelvis, measured relative to the quiet |
| | | | standing trial) |
| 9 | Velocity of the | VyP D | Anterior-posterior velocity of the pelvis subtracted from the |
| | pelvis detrended | | mean value (obtained on a step basis) |

### Movement and Orientation of the Trunk

| | | | |
|---|---|---|---|
| 1 | Anterior angle of | xTA | Flexion-extension angle of the trunk (e.g. complete stride). |
| | trunk | | Measured relative to the quiet standing trial. Negative |
| | | | represents flexion (anterior or forward lean). |
| 2 | Change in axial | ΔzTA | Axial rotation range of motion of the trunk during each stride. |
| | angle of the trunk | | zTA axial rotation of the trunk, measured relative to the quiet |
| | | | standing trial. |
| 3 | Shoulder to hip | zSH | Vertical distance between the shoulder joint centre and |
| | distance (H) | | corresponding hip joint centre (e.g. mean and range over each |
| | | | stride). |
| 4 | Shoulder to C7 | zSC7 | Vertical distance between the shoulder joint centre and |
| | distance (H) | | corresponding C7 (vertebrae) marker (mean and range over |
| | | | each stride). |
| 5 | Upper spine | USA | Mean internal angle formed by the C7, T7, and L1 vertebrae |
| | angle | | markers. Measured relative to the quiet standing trial. Negative |
| | | | represents a more acute internal angle. |
| 6 | Lower spine | LSA | Mean internal angle formed by the T7, L1, and mid-point of the |
| | angle | | two PSIS markers. Measured relative to the quiet standing trial. |
| | | | Negative represents a more acute internal angle. |

### Work Done at the Lower Limb Joints

| | | | |
|---|---|---|---|
| 1 | Hip work | HW | Positive work done at the hip joint during |
| | done | | the flight phase per unit body mass. |
| 2 | Knee work | KW | Negative work done at the knee joint during |
| | done | | the flight phase per unit body mass. |

In addition to the absolute values of each variable as described above, the length based variables (e.g. those indicated H) were also preferably normalised to standing height. These normalised variables are identified with a subscript H, e.g. zSH_{H}. Similarly, if a variable was determined at specific instance in time then these are indicated with the appropriate superscript: touchdown (TD), toe-off (TO), minimum (MIN) or maximum (MAX), e.g. VyP^{MIN}. If a value is given as a particular point within a given phase (e.g. ground contact phase (GC, the phase during which the foot of the measured leg is in contact with the ground or stance phase) or swing phase (SW, the phase during which the foot of the measured leg is not in contact with the ground or flight phase), then both the event instance and then the phase is recorded, e.g. maximum knee flexion angle during the ground contact phase would be listed as xKA MAX.GC. Mean values are over the stride cycle for a given variable. Range Δ is a range of values of a given variable from minimum to maximum. A stride is right foot (TD) to right foot TD (or left foot TD to left foot TD), a step is right foot TD to left foot TD (or left foot TD to right foot TD).

Experimental Data—Generation of a Biomechanical Model of Running

A study was carried out to investigate whether elite runners have different running kinematics to recreational runners, and to determine if there was an association between kinematic variables and running performance or economy. In addition, coach ratings of running technique were also used to compare elite and recreational runners and examine the associations between specific kinematic variables and coach ratings of technique. The study was performed in males (M) and females (F) with analysis specific to each sex, and a combined analysis (C).

Methods

Participants:

Study 1—Eighty one healthy and physically active runners who fitted the performance criteria (described below) participated in this study (males, n=42; females n=39). Study 2—The data set gathered during study 1 was expanded by the addition of a further twenty one runners to yield an overall set of 102 healthy and physically active runners (males, n=50; females, n=52). Participants were required to fit the following inclusion criteria: BMI of <24 kg·m⁻²; running >10 km per week; running to be their primary sport or activity; free from moderate/serious musculoskeletal injury in the three months prior to the test; and free from any minor musculoskeletal injury in the one month prior to the test. According to their best running performance in the previous 12 months (10 km time, or equivalent for distances between 1500 m and the marathon using IAAF points) participants were recruited to elite (M, <31; F, <35 min) and recreational groups (M, 35-50; F, 40-55 min). In addition the recreational group was further subdivided to ensure a range of running performance: fast recreational (M, 35-40; F, 40-45 min); medium recreational (M, 40-45; F, 45-50 min); and slow recreational (M, 45-50; F, 50-55 min). Individual training history (typical training volume as miles and sessions per week) and performance data were collected by questionnaire with the latter verified via official event results (Power of 10, 2015).

A panel of 10 elite coaches (8, study 2) were recruited to rate the technique of each runner at 3 velocities. 9 out of 10 had coached athletes to international competition.

Study Design:

Running participants were required to visit the laboratory on two occasions separated ≤14 days. All tests were completed in the morning (07:30-12:00) and participants were instructed to arrive at the laboratory well-hydrated, having avoided strenuous activity for 36 hrs, alcohol for 24 hrs, and caffeine ingestion for 6 hrs prior to testing. The laboratory temperature was 18-20° C.

During the first session whole body anthropometric measurements were obtained before participants performed a familiarisation run (˜30 min) on the calibrated treadmill (H/P/Cosmos, Venus T200, Nussdorf-Traunstein, Germany).

During the second session body composition was assessed using dual-energy x-ray absorptiometry (DXA). Participants then performed an incremental treadmill running test; first a sub-maximal discontinuous protocol of 4-min stages, then continuing on to a continuous protocol to exhaustion. During the sub-maximal running test there were recordings of: three-dimensional full body kinematics using an automatic motion capture system; respiratory gases to determine energy cost; two dimensional sagittal and frontal (posterior) plane video recordings that were later used by the panel of elite coaches to rate each runner's technique. In addition, the blood lactate ([La]_{b}) response to sub-maximal running was used to determine the velocity of lactate turn point (vLTP), which was the primary measure of running performance during the laboratory treadmill test, and also defined the upper boundary for the valid measurement of running economy and kinematics.

Data Collection:

Session one—A trained assessor took forty-five whole-body superficial anthropometric measurements (such as lengths and circumferences of different segments and areas of the body) on each subject according to the reduced set Yeadon inertia model (1990). Foot arch height and Achilles tendon moment arm measurements were also measured for both feet. Marks were drawn onto the most prominent medial aspect of the first metatarsal head, the most posterior aspect of the heel, and the centre point of the medial malleolus. The subject was then instructed to rest one foot on a force platform (Quattro Jump, Kistler; Winterthur, Switzerland) and the other foot on a block adjacent to the force platform, and manipulate the loading of the force platform foot to achieve 90%, 50%, and 10% of body weight. Upon attainment of a consistent loading (±1% of the target load), a sagittal plane photograph of the medial aspect of the foot was taken.

Treadmill familiarisation involved participants extensively practising mounting and dismounting the moving treadmill belt for ˜30 min. This was repeated three times at each velocity from 8 km·h⁻¹ up to the maximum of 20 km·h⁻¹ in 1 km·h⁻¹ increments, first without and then repeated whilst wearing a facemask.

Session Two:

Body mass was assessed using digital scales (Seca 700; Seca Hamburg, Germany) to the nearest 0.1 kg, and height was recorded using a stadiometer (Harpenden Stadiometer, Holtain Limited, UK). Whole-body DXA scans (Lunar iDXA; GE Healthcare, Madison, Wis., USA) were conducted whilst participants lay supine on the scanner bed and wore minimal clothing, typically running shorts and a vest. A separation between the hands and trunk, and between the legs, ensured accurate determination of body segment boundaries. All scans were performed by the same trained operator in accordance with standardised testing protocols (Nana et al. 2012; 2013; 2014; Rodriguez-Sanchez and Galloway, 2014). The iDXA was calibrated daily using the GE Lunar calibration phantom.

All participants wore running shorts and the same model of neutral racing flat training shoe (New Balance RC 1400 v2). Females were asked to wear a sports bra or tight fitting crop top, and men were asked to run without a top. Fifty six retro-reflective markers were then placed directly onto the skin of the participant where possible, and onto the shoes and tight fitting clothing where not, using double-sided tape and an adherent spray. From these, a 17-segment model of the body was defined using Visual 3D (C-Motion), from which three-dimensional joint centres and joint angles were obtained.

Prior to the start of the treadmill test, participants were instructed to adopt a modified anatomical reference position. Participants stood on the stationary treadmill belt with their feet shoulder width apart, facing directly forward, with their elbows flexed to 90°, and their forearms pointing anteriorly. A one second recording was then taken using a ten camera motion capture system (Vicon Nexus, Oxford Metrics Ltd, UK) operating at 240 Hz. Data points were averaged over the capture time period, and used as a representative recording of body configuration during quiet standing. This was used to normalise joint angles and segment orientations for the subsequent analysis. Motion capture (240 Hz) and video data (Hero4, GoPro; San Mateo, USA; 120 Hz) were recorded for 15 s after ˜30 s of each stage of submaximal running (<vLTP, see below).

Participants performed a sub-maximal, then maximal incremental running protocol with the treadmill set to level (0% incline), starting at 8 km·h⁻¹ (M) or 7 km·h⁻¹ (F), and increasing by increments of 1 km·h⁻¹. The submaximal protocol consisted of 4 min continuous running at each speed, followed by 30 s rest during which time a capillary blood sample of ˜30 μL was obtained from the fingertip for analysis of [La]_{b} (YSI 2300, Yellow Springs Instruments, Yellow Springs, Ohio). Speed increments continued until blood lactate had risen by >2 mmol·L⁻¹ from the previous stage (or exceeded 4 mmol·L⁻¹), at which point, participants entered a continuous treadmill test. In this continuous test the treadmill speed increased by 1 km·h⁻¹ every 2 min until volitional exhaustion.

Breath-by-breath gas exchange and ventilation rates were measured continuously throughout the incremental treadmill test. Participants wore a low-dead space mask and breathed through an impeller turbine assembly (Jaeger Triple V, Jaeger, Hoechberg, Germany). The inspired and expired gas volume and concentration signals were continuously sampled, the latter using paramagnetic (O₂) and infrared (CO₂) analysers (Jaeger Vyntus CPX, Carefusion, San Diego, Calif.) via a capillary line. Before each test, the gas analysers were calibrated via a two point calibration with gases of known concentration (16% O₂ and 5% CO₂) and ambient air, and the turbine volume transducer was calibrated using a 3 L syringe (Hans Rudolph, Kansas City, Mo.). The volume and concentration signals were time aligned, accounting for the transit delay in capillary gas and analyser rise time relative to the volume signal.

Data Analysis

Anthropometrics—

Superficial anthropometric measurements were inputted to the Yeadon (1990) inertia model software (Inertia, Visual 3D, C-Motion, Oxford, UK) to generate subject-specific segmental inertia parameters for each body segment of each subject. This data was exported as a script file for subsequent use in the analysis conducted using Visual 3-D software (C-Motion; Maryland, USA).

Foot photographs were digitised to give dorsal height (DH) (height of the foot, halfway along the foot length), and truncated foot length (TFL) (distance between the most prominent medial aspect of the first metatarsal head and the most posterior aspect of the heel). From these, Arch Height Index (AHI) was calculated as (Pohl & Farr, 2010):

AHI=DH/TFL (1)

Relative arch deformity (RAD) was calculated using the Arch Height Index of the unloaded foot at 10% body weight (AHIU), the loaded foot at 90% body weight (AHIL); and body mass (BM) (Williams and McClay, 2000):

RAD=((AHIU−AHIL)/AHIU)*(10⁴/BM) (2)

Achilles tendon moment arm (ATMAL) was calculated as the perpendicular distance between the centre of the medial malleolus, and the line longitudinally bisecting the belly of the Achilles tendon, as palpated and identified by one of the researchers.

The iDXA software allowed whole-body and segmental (foot, shank, thigh) proportional composition and mass (fat, lean, and bone) to be determined, as well as segment lengths.

Pulmonary gas exchange—

Breath-by-breath \dot{V}O₂ data were initially examined to exclude errant breaths caused by coughing, swallowing, etc., and those values lying more than 4 SD from the local mean were removed. Subsequently, the breath-by-breath data were converted to second-by-second data using linear interpolation. Oxygen consumption (\dot{V}O₂), carbon dioxide production (\dot{V}CO₂), ventilation rate (\dot{V}_{E}), and the respiratory exchange ratio (RER) were quantified for the final 60 s of each stage of the submaximal protocol. \dot{V}O₂ peak was determined as the highest 30 s moving average.

Velocity of Lactate Turn Point (vLTP)

vLTP was identified via a derivation of the modified Dmax method (Bishop et al. 1998). Briefly, a fourth order polynomial curve was fitted to the speed-lactate relationship. Lactate threshold (LT) was identified as the first stage with an increase in [La]_{b}>0.4 mmol·L⁻¹ above the baseline level (moving average of 3 lowest values) and a straight line was drawn between LT and the last 4-min stage of running (i.e. a rise of >2 mmol·L⁻¹ or exceeding 4 mmol·L⁻¹). Finally vLTP was defined as the greatest perpendicular distance between this straight line and the fourth order polynomial, to the nearest 0.5 km·h⁻¹. For the combined analysis of males and females, sex independent values were also calculated for each individual as a z-score (difference in standard deviations of their vLTP from the sex-specific mean).

### Running Economy

The average second-by-second \dot{V}O₂ and \dot{V}CO₂ during the final minute of each submaximal stage were used to calculate the energy cost (E_{c}) of running. Updated non-protein respiratory quotient equations (Peronnet and Massicotte, 1991) were used to estimate substrate utilisation (g·min⁻¹). The energy derived from each substrate was calculated by multiplying fat and carbohydrate utilisation by 9.75 and 4.07 kilocalories (kcal), respectively (Jeukendrup and Wallis, 2005). Absolute E_{c} was calculated as the sum of the energy derived from fat and carbohydrate for each speed up to the stage before vLTP (assuming a RER value of <1.00), in order to ensure an insignificant contribution of anaerobic metabolism to energy expenditure, and expressed in kcal per kg body mass per km (kcal·kg⁻¹·km⁻¹). At the end of study 2, energy expenditure at rest was subtracted from the running measurements to calculate the energy cost (Ec) of running and calculated for each velocity 9-17 km·hr⁻¹. Subsequently data were averaged for slow (9, 10 & 11 km·hr⁻¹), medium (12 & 13 km·hr⁻¹) and fast (14 & 15 km·hr⁻¹) velocities of running.

### Kinematic Variables

The raw marker data was initially labelled in Vicon Nexus with a combination of spline and pattern filling used to fill gaps in the trajectories (the maximum filled gap length was set to 10 frames). A bidirectional butterworth filter (4th order) with a cut-off frequency of 15 Hz was applied to individual marker trajectories, with the frequency selected based on a residual analysis of marker trajectories (Winter, 1990). The labelled and filtered data were then exported to Visual 3D (v5.01, C-Motion, Oxford, UK) where a 17-segment model of the body was created. Marker location, and segment, joint centre and joint angle time histories were then exported to Matlab (R2015b, Mathworks; Massachusetts, USA) where algorithms were developed to calculate each of the key variables. A total of 34 variables were calculated and subsequently divided into six sub-groups: temporal-spatial parameters; lower limb joint and segment angles; movement of the centre of mass; movement and orientation of the pelvis; movement and orientation of the trunk; and work done at the lower limb joints.

The instants of touchdown and toe-off for each step based on the accelerations and jerk of the heel, first metatarsal head and hallux markers were automatically detected using an algorithm developed within Matlab. This algorithm was based on previously published methods for detecting touchdown and toe-off during treadmill running using foot marker data (Giandolini et al. 2014; Leitch et al. 2011; and Maiwald et al. 2009). The modified algorithm emerged from a laboratory-based evaluation of the previous methods in which a number of participants ran over-ground over a force platform utilising a range of footstrike types. In brief, touchdown was found to be most accurately determined as the time of the vertical acceleration peak of the heel or first metatarsal head marker (whichever occurred first) while toe-off was determined as the time of the vertical jerk peak of the hallux marker. The algorithm allowed touchdown and toe-off to be detected to within 3.75±4.92 ms and 1.51±7.60 ms respectively.

Based on these events the time series data was split into step (right foot touchdown to left foot touchdown) and stride (right foot touchdown to right foot touchdown) cycles. Thereafter, the values of each of the kinematic variables at these key instances, or maxima/minima within each step or stride could be obtained. Where relevant, the variable was expressed relative to the quiet standing trial value. All 34 variables were calculated for 10 stride cycles and then the mean and standard deviation obtained for group analysis. This process was repeated for each subject at each running speed up to and including their lactate turnpoint speed. In addition, all kinematic length-dependent variables were normalised to participant standing height, and mass-dependent variables to participant body mass. At the end of study 2, kinematic data for all measured variables were calculated for each velocity between 9 and 17 km·hr⁻¹. Subsequently data were averaged for slow (9, 10 & 11 km·hr⁻¹), medium (12 & 13 km·hr⁻¹) and fast (14 & 15 km·hr⁻¹) velocities of running.

### Coach Ratings

The sagittal and frontal plane video recordings underwent a two-step process prior to being given to the coaches for technique rating. Individual videos of the frontal (posterior) and sagittal (left) views were initially cropped in GoPro Studio (v2.0), so that only the participant and the treadmill were in view (8:9 aspect). They were then exported into Stereo Movie Maker (v1.3), where the videos were cut to ˜6s, starting and finishing with a right foot toe-off, to facilitate a continuous looping of the video. The two simultaneous videos of the sagittal and frontal views were then placed together into one video of widescreen 16:9 aspect, and exported as AVI files. The participants' identity was obscured by using the posterior view of the participant, and by the facemask in the sagittal view. The coaches were provided with three video clips of each participant one at each of three speeds (F 11, 13 and 15 km·h⁻¹; M 13, 15 and 17 km·h⁻¹). Video clips were presented in a randomised and anonymised order, both between and within participants. The coaches scored the runner in each video clip on their overall technique and 12 specific technical variables. Thirty video clips (10 runners at the same speeds) were duplicated within the anonymised list of video clips given to the coaches in order to assess the reliability of the coach ratings. The ratings from the 10 coaches were averaged to generate representative values of the coaching panel. The average of the coach ratings of overall technique for each runner were used to define coach rated high (CR high, n=10) and low (CR low, n=10) groups with male, female and combined cohorts in order to compare the kinematics of these groups.

### Statistical Analysis

Data are presented as mean±sd. For the combined cohort (i.e. males and females) analysis of covariance (ANCOVA) with sex as the covariant was conducted to assess the effect of group (Elite vs Recreational; CR high vs CR low) on kinematic variables at each running velocity (slow, medium, high), as well as six selected anthropometric and physiological parameters. Additional independent t-tests were performed to test the effect of group (Elite vs Recreational; CR high vs CR low) on kinematic variables in M and F separately at each running velocity. Statistical significance was accepted at p ≤0.05 level, and tendencies towards statistical significance were identified if p ≤0.10. All statistical analysis procedures were performed with IBM SPSS Statistics Version 21 (IBM Corp., New York, N.Y., USA).

The relationship between kinematic variables measured at each velocity (study 1: 9, 11, 13, 15 km·hr⁻¹; study 2: slow, medium, fast) and outcome variables including absolute performance (M/F: 10 km time, vLTP), sex-independent performance (C: IAAF points, vLTPz), running economy (Ec at each velocity) and coach ratings (overall technique rating at each velocity) were first assessed as bivariate relationships with independent Pearson's product moment correlations. Study 1: Individual correlation coefficients at each velocity (9, 11, 13, 15 for M; 9, 11, 13 for F) were averaged and these averaged correlation coefficients were used to determine significance of bivariate relationships. Kinematic variables that produced significant average correlation coefficients (typically 3-8 variable per outcome) were then included in stepwise and/or forced entry multiple linear regression. A 4% improvement in the explained variance was used as a threshold for the inclusion of additional factors. Study 2: The relationship between kinematic variables measured at each velocity (slow, medium, fast) and outcome variables including absolute performance (M/F: 10 km time, vLTP), sex-independent performance (C: vLTPz), running economy (Ec at each velocity) and coach ratings (CR of overall technique) were first assessed as bivariate relationships with independent Pearson's product moment correlations. This was done for each velocity category and cohort (slow, medium for F & C; slow, medium & fast for M). The kinematic variables significantly correlated within an outcome (Ec, vLTP, vLTPz, CR) were subsequently included within a stepwise multiple linear regression to calculate the greatest possible variance of the outcome explained by addition of significant variables. In addition forced entry multiple linear regression was performed with 2 or 3 selected variables of interest that were consistently correlated with the outcome.

### Results—Study 1

### Overview of the Kinematic Differences Between Elite Vs Recreational Runners

Table 1 shows the kinematic variables with significant differences or tendencies between recreational and elite runners at 9, 11 and 13 km·h⁻¹ in the combined (M and F) ANCOVA. P values are based on t-tests between elite and recreational. Bold indicates P<0.05.

| | | Velocity | | | | | | | | | | | |
|---|---|---|---|---|---|---|---|---|---|---|---|---|---|
| Kinematic | | 9 | | | | 11 | | | | 13 | | | |
| Variable | Group | N | Mean | SD | P | N | Mean | SD | Sig. | N | Mean | SD | P |

Ground contact variables and leg configuration at touchdown:

| | | | | | | | | | | | | | |
|---|---|---|---|---|---|---|---|---|---|---|---|---|---|
| GCT (s) | Elite | 24 | 0.259 | 0.026 | .007 | 25 | 0.234 | 0.019 | .002 | 25 | 0.214 | 0.018 | .003 |
| | Rec | 54 | 0.276 | 0.027 | | 55 | 0.249 | 0.022 | | 45 | 0.227 | 0.019 | |
| DF (—) | Elite | 24 | 0.357 | 0.043 | .073 | 25 | 0.326 | 0.035 | .007 | 25 | 0.305 | 0.032 | .002 |
| | Rec | 54 | 0.373 | 0.037 | | 55 | 0.347 | 0.027 | | 45 | 0.326 | 0.023 | |
| TD-CM (m) | Elite | 24 | 0.315 | 0.029 | .056 | 25 | 0.341 | 0.029 | .032 | 25 | 0.364 | 0.030 | .044 |
| | Rec | 50 | 0.327 | 0.031 | | 51 | 0.355 | 0.031 | | 43 | 0.378 | 0.034 | |
| TD-CM_{H} (—) | Elite | 24 | 0.181 | 0.018 | .037 | 25 | 0.196 | 0.018 | .017 | 25 | 0.209 | 0.018 | .026 |
| | Rec | 50 | 0.191 | 0.018 | | 51 | 0.208 | 0.018 | | 40 | 0.220 | 0.019 | |
| TO-CM (m) | Elite | 24 | −0.286 | 0.033 | .028 | 25 | −0.322 | 0.030 | .013 | 25 | −0.351 | 0.031 | .024 |
| | Rec | 50 | −0.302 | 0.035 | | 51 | −0.339 | 0.030 | | 43 | −0.366 | 0.030 | |
| TO-CM_{H} (—) | Elite | 24 | −0.164 | 0.018 | .012 | 25 | −0.185 | 0.015 | .002 | 25 | −0.201 | 0.016 | .004 |
| | Rec | 50 | −0.176 | 0.020 | | 51 | −0.198 | 0.016 | | 40 | −0.214 | 0.016 | |
| GCD (m) | Elite | 24 | 0.600 | 0.056 | .018 | 25 | 0.663 | 0.051 | .008 | 25 | 0.715 | 0.053 | .019 |
| | Rec | 50 | 0.629 | 0.058 | | 51 | 0.693 | 0.055 | | 40 | 0.745 | 0.058 | |
| GCD_{H} (—) | Elite | 24 | 0.344 | 0.031 | .008 | 25 | 0.380 | 0.028 | .002 | 25 | 0.410 | 0.029 | .004 |
| | Rec | 50 | 0.368 | 0.033 | | 51 | 0.405 | 0.030 | | 40 | 0.434 | 0.031 | |
| ΔSA (°) | Elite | 24 | 35.1 | 6.3 | .063 | 25 | 37.2 | 5.5 | .015 | 25 | 39.9 | 5.2 | .014 |
| | Rec | 54 | 37.8 | 5.2 | | 55 | 40.3 | 4.6 | | 46 | 43.0 | 4.6 | |
| FSA (°) | Elite | 24 | 3.0 | 8.8 | .017 | 25 | 3.7 | 8.6 | .007 | 24 | 5.3 | 9.3 | .008 |
| | Rec | 54 | 8.6 | 9.2 | | 55 | 10.3 | 9.7 | | 45 | 12.1 | 10.0 | |
| AA_{TD} (°) | Elite | 24 | −2.5 | 7.7 | .052 | 25 | −2.9 | 7.4 | .040 | 25 | −2.7 | 7.6 | .021 |
| | Rec | 54 | 1.5 | 8.4 | | 55 | 1.3 | 8.6 | | 45 | 2.0 | 8.2 | |
| HA_{TD} (°) | Elite | 24 | 23.8 | 3.3 | .007 | 25 | 26.5 | 3.3 | .027 | 24 | 28.4 | 3.2 | .040 |
| | Rec | 54 | 27.9 | 6.4 | | 55 | 29.9 | 6.7 | | 44 | 31.7 | 7.2 | |
| SA_{TD} (°) | Elite | 24 | 6.1 | 3.0 | .053 | 25 | 7.3 | 3.0 | .006 | 25 | 9.0 | 3.2 | .016 |
| | Rec | 54 | 7.9 | 3.7 | | 55 | 9.9 | 3.8 | | 46 | 11.1 | 3.6 | |

Leg configuration at mid-stance:

| | | | | | | | | | | | | | |
|---|---|---|---|---|---|---|---|---|---|---|---|---|---|
| KA_{MIN} (°) | Elite | 24 | −36.1 | 3.7 | .046 | 25 | −37.7 | 3.6 | .089 | 25 | −38.7 | 3.4 | .055 |
| | Rec | 54 | −39.0 | 6.5 | | 55 | −40.1 | 6.5 | | 45 | −41.4 | 6.4 | |

Pelvis and trunk axial movements, and lower (lumbar) spine posture:

| | | | | | | | | | | | | | |
|---|---|---|---|---|---|---|---|---|---|---|---|---|---|
| ΔzPA (°) | Elite | 24 | 10.5 | 2.9 | .079 | 25 | 12.3 | 3.5 | .038 | 24 | 13.7 | 3.7 | .038 |
| | Rec | 54 | 12.4 | 4.3 | | 55 | 14.7 | 4.7 | | 44 | 16.3 | 5.3 | |
| ΔzTA (°) | Elite | 24 | 19.0 | 4.4 | .058 | 25 | 21.6 | 4.3 | .015 | 25 | 23.5 | 4.8 | .009 |
| | Rec | 54 | 22.0 | 6.4 | | 55 | 25.8 | 7.2 | | 45 | 27.6 | 7.3 | |
| LSA_{MEAN} (°) | Elite | 22 | 1.6 | 4.6 | .123 | 23 | 1.5 | 4.5 | .055 | 22 | 0.9 | 4.3 | .040 |
| | Rec | 51 | 3.5 | 4.8 | | 49 | 4.0 | 5.3 | | 38 | 3.8 | 5.5 | |
| ΔLSA (°) | Elite | 22 | 5.1 | 2.5 | .029 | 23 | 5.6 | 2.63 | .027 | 22 | 6.1 | 3.0 | .011 |
| | Rec | 51 | 6.8 | 3.4 | | 49 | 7.5 | 3.82 | | 38 | 8.4 | 4.4 | |

Swinging leg:

| | | | | | | | | | | | | | |
|---|---|---|---|---|---|---|---|---|---|---|---|---|---|
| HW_{POS} | Elite | 24 | 0.414 | 0.127 | .001 | 24 | .551 | 0.146 | .010 | 25 | 0.722 | 0.176 | .043 |
| (J · kg⁻¹) | Rec | 52 | 0.332 | 0.086 | | 52 | .476 | 0.095 | | 42 | 0.652 | 0.111 | |

Ground Contact Variables and Leg Configuration at Touchdown:

The recreational runners had a longer ground contact time than elite runners (GCT+17, +16 and +13 ms at 9, 11 and 13 km·h⁻¹), a greater ground contact distance (GCD, ˜3 cm longer at 9-13 km·h⁻¹), a higher duty factor (DF, +0.017 up to +0.023 for 9-13 km·h⁻¹) and a greater shank angle range of motion during ground contact (ΔSA_{GC}, at ˜3° at 9-13 km·h⁻¹). Both components of the GCD, touchdown in front of the CM (GCD_{TD}, TD-CM) and toe-off behind the CM (GCD_{TO}, TD-CM) were greater in recreational runners. Notably, although the recreational runners spent longer in ground contact, there was no difference in flight (swing) times. The M only comparisons produced supporting significant differences across the same range of variables, while the F only comparisons produced supporting significant differences in GCT only.

Leg Configuration at Mid-Stance:

The recreational runners tended to sink to a lower position at mid-stance than elite runners; indicated by greater peak knee flexion during ground contact (KA_{MIN}, ˜3° at 9-13 km·h⁻¹). The F only comparisons produced a supporting significant difference in KA_{MIN}.

Pelvis and Trunk Axial Movements, and Lower (Lumbar) Spine Posture:

Recreational runners had a greater range of axial rotation of the pelvis (ΔzPA, +2° to +3° for 9-13 km·h⁻¹) and the trunk (ΔzTA, +3° to +4° for 9-13 km·h⁻¹). The F only comparisons produced supporting significant differences for the trunk axial rotation.

Recreational runners also had a more extended lower spine on average throughout the stride cycle (LSA_{MEAN}, +2° to +3° for 9-13 km·h⁻¹), as well as a greater range of lower spine movement (ΔLSA, +2° for 9-13 km·h⁻¹) compared to elite runners. The M only comparisons produced supporting significant differences in LSA.

Swinging Leg:

Recreational runners performed less positive hip work during the swing phase compared to the elite runners; indicated by a lower work done at the hip during swing (HW_{POS}, −0.08 to −0.07 J·kg⁻¹ for 9-13 km·h⁻¹). The F only comparisons produced supporting significant differences for HW.

### Overview of the Relationship Between Kinematic Variables and Running Performance, Economy and Coach Ratings.

### Correlation Analysis:

For each outcome parameter there were between 3 and 8 kinematic variables that were significantly related to the outcome, as shown by significant average correlation coefficients across 3 or 4 measurement velocities (Table 2). These average correlation coefficients were typically weak to moderate in nature ranging from r=0.25 to r=0.46.

TABLE 2
Significant average correlation coefficients between
running performance an economy outcome measures and kinematic
variables measured at a range of 3/4 speeds.

| | Kinematic Variable: | vLTP | 10k time | Ec |
|---|---|---|---|---|
| Males | GCT | −.381 | .354 | |
| | GCD | −.385 | .448 | |
| | TO − CM | .369 | −.409 | |
| | TD − CM | −.322 | .392 | |
| | VyP (MIN) | .398 | | −0.45 |
| | Δ VyP | | | 0.42 |
| | Δ VyCM | | | 0.43 |
| | Δ SA | | .373 | |
| | LSA (MEAN) | | .334 | |

Measured across velocities of 9, 11, 13, 15 km · h⁻¹

| | Kinematic Variable: | vLTP | 10k time | Ec |
|---|---|---|---|---|
| Females | VyP (MIN) | .385 | −.339 | −0.37 |
| | Δ zP | | | 0.362 |
| | xPA (MEAN) | .458 | −.373 | −0.347 |
| | HA (TD) | −.426 | .367 | |
| | xTA (MEAN) | .382 | −.349 | |
| | Δ zTA | −.397 | | |
| | HW | .391 | −.402 | |

Measured across velocities of 9, 11, 13 km · h⁻¹

| | Kinematic Variable: | vLTPz | IAAF Points | Ec |
|---|---|---|---|---|
| Combined | GCT | −.278 | −0.29 | |
| | TO − CM | .246 | | |
| | Δ zP | | | 0.384 |
| | Δ VyP | −.309 | | 0.411 |
| | VyP (MAX) | | | 0.305 |
| | VyP (MIN) | .412 | 0.28 | −0.457 |
| | Δ zCM | | | 0.311 |
| | Δ VyCM | | | 0.298 |
| | VyCM (MAX) | −.255 | −0.29 | |
| | Δ zPA | | | 0.428 |
| | KA (PEAK) | | | −0.355 |
| | Δ zTA | −.307 | | |

Measured across velocities of 9, 11, 13, 15 km · h⁻¹

The minimum horizontal velocity of the pelvis (VyP^{MIN}), was significantly related to 8 of the 9 running performance and energy cost outcome parameters across M, F and C cohorts. In fact this was the only variable related to running performance and economy in M and F, as other kinematic variables were more sex specific. For example, hip, pelvis and trunk position and movement variables were commonly associated with running performance/economy outcomes in F (HW, xPA, xTA, ΔzTA, ΔzP_{H}), but not in M. In contrast ground contact parameters (GCT, GCD_{H}, ΔSA, TO-CM_{H}, TD-CM_{H}) were often significantly associated with performance and economy in M.

For the combined data the significant kinematic variables appeared to be more reflective of the pelvis and trunk position and movements, although some CM movement variables also became uniquely apparent within this cohort. Given the greater range of data and larger numbers it might have been expected that the combined analysis would reveal stronger relationships between kinematic variables and running economy/performance. However this was not the case, likely due to the apparent sex specificity of many of the relationships.

A larger number of kinematic variables were related to the overall coach rating of running technique (4-12 for the three cohorts; Table 3) with weak, moderate and strong coefficients ranging from r=0.30 to r=0.81 that were typically higher than for the other outcome measures e.g. running performance or economy. Some of the significant variables were similar for M, F and C i.e. FLT, DF, ΔzP_{H}. FLT in particular was moderately to strongly correlated with coach ratings r=0.59-0.81 for the different cohorts (M, F, C).

TABLE 3
Significant average correlation coefficients between coaching
ratings and kinematic variables both measured at 2 (F,
C) or 3 (M) speeds. Kinematic variables and coach ratings
were measured across 2 or 3 velocities of running.

| | Coach rating for 3 cohorts | | |
|---|---|---|---|
| Kinematic variable | M | F | C |
| GCD_{H} | −0.567 | | −0.432 |
| GCT | −0.537 | | −0.322 |
| FLT | 0.591 | 0.806 | 0.667 |
| DF | −0.698 | −0.659 | −0.653 |
| TO − CM | 0.633 | | |
| FSA | −0.487 | | −0.360 |
| AA_{TD} | −0.465 | | |
| SA_{TD} | | | −0.433 |
| SA_{TO} | 0.441 | | 0.302 |
| ΔSA | −0.545 | | −0.502 |
| VyP^{MAX} | | | 0.438 |
| ΔzCM_{H} | | 0.735 | 0.608 |
| ΔVyP | | | 0.421 |
| ΔzP_{H} | 0.485 | 0.719 | 0.601 |
| ΔzCM_{H} | 0.494 | | |

In females VyP^{MIN} combined with HW explained 26% of the variance in 10 km time and 30% of the variance in vLTP during the laboratory treadmill running test (Table 4). In males VyP^{MIN} and GCT explained 28% of the variance in vLTP, however 25% of the variance in 10 km time was predicted by GCD_{H} (the spatial equivalent of GCT, normalised to height) and LSA. Within each sex group similar predictors explained performance of vLTP and 10 km time, which would seem to corroborate the importance of these predictor variables in each case. Moreover, in a similar manner as for the correlation analysis there was a degree of sex specificity within the kinematic predictor variables, with ground contact time/distance important for M and hip work important for F.

Considering vLTP as the most experimentally robust measure of performance in this study (because it was measured on the same day and during the same test and conditions as the kinematic measures) VyP^{MIN} was an important predictor of performance in both M and F. This was also the case for the combined analysis of performance (outcomes: IAAF points and vLTPz) where VyP^{MIN} and GCT explained 19 and 24% of the variance respectively.

The energy cost of running for M, F and C was explained by VyP^{MIN} in combination with another variable (M, ΔVyCM; F, ΔzP_{H}; C, ΔzPA) accounting for 26, 17 and 22% of the variance.

Overall VyP^{MIN} was a predictive factor in 8 of the 9 regressions for explaining running performance or economy outcome measures.

Flight time (FLT) explained 50% (F) and 44% (C) of the variance in the overall coaching rating of technique. Within M, the similar variable of duty factor (DF, the proportion of time the runner is in the air) in combination with TO-CM explained 53% of the variance in coach ratings. Therefore kinematic factors related to the time the athlete is in the air explained a large proportion of what the coaches considered good technique.

Furthermore qualitatively kinematic variables explained a greater proportion of the variance in the coach ratings than running economy or performance.

TABLE 4
Multiple regression analysis showing the one/two kinematic variables
that in combination explained the largest variance of the outcome.

| | | | | Total (%) variance |
|---|---|---|---|---|
| | | Kinematic predictors | | explained |
| Outcome | Group | Var. 1 | Var. 2 | mean (range) |
| 10k time | M | GCD_{H} | L̅S̅A̅ | 25 (20-29) |
| | F | VyP^{MIN} | HW | 26 (16-33) |
| IAAF pts | C | VyP^{MIN} | GCT | 19 (17-22) |
| vLTP | M | VyP^{MIN} | GCT | 28 (20-35) |
| | F | VyP^{MIN} | HW | 30 (22-36) |
| vLTPz | C | VyP^{MIN} | GCT | 24 (22-27) |
| E_{C} | M | VyP^{MIN} | ΔVyCM | 26 (19-35) |
| | F | VyP^{MIN} | ΔzP_{H} | 17 (6-24) |
| | C | VyP^{MIN} | ΔzPA | 22 (18-28) |
| CR | M | DF | TO − CM | 53 (37-65) |
| | F | FLT | — | 50 (42-55) |
| | C | FLT | — | 44 (35-53) |

### Results—Study 2

### Kinematic Differences Between Elite Vs Recreational Runners

(Significant differences are reported in brackets as recreational vs elite for slow and medium velocities).

### Temporal Variables

Two variables relating to the proportion of time runners were either in contact with the ground or airborne were significantly different between elite and recreational runners. The recreational runners had a longer ground contact time than elite runners (GCT: 26.2 ms vs 24.5 ms; 23.3 ms vs 21.8 ms), and a higher duty factor (DF: 35.7% vs 34.0%; 32.9% vs 31.0%). There was no difference in flight time between recreational and elite runners so the greater DF in recreational runners was largely due to their longer GCT.

### Ground Contact Variables

Positions—The recreational runners exhibited a greater normalised ground contact distance (GCD_{H}: 0.386 vs 0.364; 0.428 vs 0.404), this result was corroborated by larger normalised distances between the CM and the foot at touch down (TD-CM_{H}: 0.200 vs 0.189; 0.218 vs 0.207) and toe off (TO-CM_{H}: −0.187 vs −0.174; −0.210 vs −0.197). The equivalent variables relating to the pelvis position rather than the CM all showed similar differences. During ground contact the pelvis of the recreational athletes tended to reach a lower normalised minimum height (zP_{H} MIN: −0.041 vs −0.037; −0.044 vs −0.041). This could be indicative of recreational athletes wasting energy by moving their CM through an unnecessarily large vertical range.
Velocities—There were differences in the horizontal velocity of the CM between the two groups; the maximum CM velocity was higher in recreational athletes than elite athletes (VyCM^{MAX}: 0.059 m·s1 vs 0.048 m·s1; 0.062 m·s1 vs 0.050 m·s1). Since mean CM velocity should be close to zero whilst running at a constant velocity on a treadmill this indicates that recreational athletes may have been going through a greater velocity range than elite athletes, which is indicative of a greater level of braking and subsequent acceleration.

### Angles of the Leg

A number of joint angle differences were apparent between the two groups in the legs. The foot strike angle was significantly higher in recreational runners indicating that they tended to be heel strikers, and that the elite athletes tended to be mid-foot strikers (xFA TD: 10.0° vs 3.5°; 12.0° vs 5.6°). In addition to the foot strike angle, the ankle was also slightly dorsiflexed in recreational runners and slightly plantar flexed in elite runners at touchdown (xAA TD: 2.1° vs −2.5°; 2.2° vs −2.4°). Differences were also observed in the shank angle; this was more positively oriented (leant back) at touchdown (xSA TD: 8.8° vs 6.8°; 10.6° vs 8.9°), and moved through a larger range during stance (ΔxSA GC: 39.0° vs 35.9°, and 42.6° vs 39.5°) in recreational athletes. The knee angle also showed differences between the two groups, it was flexed more during ground contact in the recreational runners which is likely to have led to the lower pelvis position identified above (xKA MIN GC: 40.4° vs 37.1°, and 42.0° vs 38.7°) and the increased angular range through which the shank traveled. The hip angle at touchdown was more flexed in recreational athletes (e.g. the foot was further in front of the body) (xHA TD: 28.5° vs 25.1°; 31.1° vs 27.9°), which ties in with the increased shank, foot, and ankle angles, and the increased foot to CM distance described above. The overall picture is one of recreational athletes contacting the ground with the foot well in front of the body, leading to a reduction in CM velocity and flexion at the knee joint. This absorbs energy that then has to be re-generated in the push off phase in order to maintain constant velocity. This requires more energy generation in the muscles of the stance leg which is inefficient and could lead to premature fatigue.

### Angles of the Pelvis, Spine and Trunk

In addition to the joint angles of the leg, a number of the angles of the upper body were also significantly different between elite and recreational runners. The most consistent differences were in the ranges through which the joint angles moved. The ranges of pelvis and trunk rotations about the long axis of the body were significantly higher in recreational athletes (ΔzPA: 13.5° vs 11.3°; 15.7° vs 13.4°) (ΔzTA: 23.7° vs 20.1°; 26.6° vs 23.0°; 29.9° vs 23.8°). In addition to these ranges, the minimum pelvis angle was significantly more negative (indicating more anterior tilt in relation to standing) in recreational athletes (xPA MIN: −9.9° vs −7.6°; −11.1° vs 9.1°; −11.0° vs 10.1°). The lower spine angle also went through a greater range in recreational athletes (ΔLSA: 7.1° vs 5.2°; 8.2° vs 6.0°; 9.0° vs 6.5°). Overall recreational athletes seem to rotate more in various parts of the body and about various axes than elite athletes. This could indicate wasteful motions which cause energy to be expended in rotational actions which do not contribute to the forward velocity of the CM. It is clear that having a stable pelvis and trunk is a feature of the techniques of elite athletes and that this is likely to benefit performance.

### Hip Work

The amount of work done by the hip during the swing phase was significantly higher in the elite group than the recreational (HW: 0.41 J·km¹ vs 0.47 J·km¹; 0.60 J·km¹ vs 0.68 J·km¹); this is likely to be due to a more forceful flexion-extension of the hip, leading to a higher angular velocity at touchdown which could limit the braking effect of the stance leg.

TABLE 5
Kinematic variables with significant differences or tendencies between
recreational (R) and elite (E) runners at slow (9, 10 & 11 km/hr), medium
(12 & 13 km/hr) running speeds in the combined (M and F) ANCOVA.

| | Slow | | | | Medium | | | |
|---|---|---|---|---|---|---|---|---|
| Kinematic Variable: | | Mean ± St Dev | n | p = | | Mean ± St. Dev | n | p = |

Time

| | | | | | | | | |
|---|---|---|---|---|---|---|---|---|
| GCT | E | 0.245 ± 0.021 | 29 | 0.001 | E | 0.218 ± 0.017 | 27 | 0.001 |
| | R | 0.262 ± 0.025 | 73 | | R | 0.234 ± 0.020 | 63 | |
| DF | E | 0.340 ± 0.036 | 28 | 0.015 | E | 0.310 ± 0.031 | 27 | 0.001 |
| | R | 0.357 ± 0.030 | 73 | | R | 0.329 ± 0.023 | 63 | |

Positions and Velocities

| | | | | | | | | |
|---|---|---|---|---|---|---|---|---|
| TD-CM_{H} | E | 0.189 ± 0.018 | 29 | 0.011 | E | 0.207 ± 0.020 | 27 | 0.007 |
| | R | 0.200 ± 0.018 | 70 | | R | 0.218 ± 0.017 | 59 | |
| TO-CM_{H} | E | −0.174 ± 0.015 | 29 | 0.002 | E | −0.197 ± 0.014 | 27 | 0.00 |
| | R | −0.187 ± 0.018 | 70 | | R | −0.210 ± 0.016 | 59 | |
| GCD(CM)_{H} | E | 0.364 ± 0.029 | 29 | 0.001 | E | 0.404 ± 0.029 | 27 | 0.00 |
| | R | 0.386 ± 0.031 | 70 | | R | 0.428 ± 0.029 | 59 | |
| TD-PEL_{H} | E | 0.159 ± 0.017 | 29 | 0.035 | E | 0.177 ± 0.019 | 27 | 0.02 |
| | R | 0.168 ± 0.020 | 73 | | R | 0.187 ± 0.020 | 62 | |
| TO-PEL_{H} | E | −0.205 ± 0.016 | 29 | 0.003 | E | −0.228 ± 0.015 | 27 | 0.001 |
| | R | −0.217 ± 0.018 | 73 | | R | −0.240 ± 0.016 | 62 | |
| GCD(PEL)_{H} | E | 0.364 ± 0.028 | 29 | 0.002 | E | 0.405 ± 0.028 | 27 | 0.001 |
| | R | 0.385 ± 0.031 | 73 | | R | 0.427 ± 0.030 | 62 | |
| zP_{H} MIN | E | −0.037 ± 0.005 | 29 | 0.007 | E | −0.041 ± 0.005 | 27 | 0.029 |
| | R | −0.041 ± 0.007 | 73 | | R | −0.044 ± 0.007 | 62 | |
| ΔzP_{H} GC | E | 0.046 ± 0.007 | 29 | 0.021 | E | 0.041 ± 0.006 | 27 | 0.125 |
| | R | 0.049 ± 0.007 | 73 | | R | 0.044 ± 0.006 | 62 | |
| VyCM MAX | E | 0.048 ± 0.013 | 28 | 0.00 | E | 0.050 ± 0.017 | 26 | 0.007 |
| | R | 0.059 ± 0.015 | 68 | | R | 0.062 ± 0.018 | 57 | |

Angles and Work done

| | | | | | | | | |
|---|---|---|---|---|---|---|---|---|
| xFA TD | E | 3.528 ± 8.219 | 29 | 0.001 | E | 5.594 ± 8.831 | 27 | 0.003 |
| | R | 10.025 ± 8.922 | 73 | | R | 11.983 ± 9.286 | 86 | |
| xAA TD | E | −2.494 ± 7.025 | 29 | 0.01 | E | −2.446 ± 7.173 | 27 | 0.012 |
| | R | 2.051 ± 8.070 | 73 | | R | 2.230 ± 8.068 | 63 | |
| xSA TD | E | 6.783 ± 3.032 | 29 | 0.006 | E | 8.883 ± 3.245 | 27 | 0.012 |
| | R | 8.801 ± 3.443 | 73 | | R | 10.641 ± 3.400 | 63 | |
| xSA TO | E | −29.001 ± 4.190 | 29 | 0.243 | E | −30.381 ± 3.509 | 27 | 0.11 |
| | R | −30.111 ± 4.445 | 73 | | R | −31.798 ± 3.870 | 63 | |
| ΔxSA GC | E | 35.921 ± 5.540 | 29 | 0.007 | E | 39.455 ± 5.155 | 27 | 0.003 |
| | R | 39.034 ± 4.803 | 73 | | R | 42.552 ± 4.400 | 63 | |
| xKA MIN.GC | E | −37.130 ± 3.949 | 29 | 0.012 | E | −38.704 ± 3.805 | 27 | 0.01 |
| | R | −40.353 ± 6.202 | 73 | | R | −41.986 ± 6.221 | 63 | |
| yKA TD | E | 0.415 ± 1.751 | 28 | 0.061 | E | 0.601 ± 1.822 | 25 | 0.047 |
| STAND | R | −0.355 ± 1.812 | 72 | | R | −0.243 ± 1.918 | 61 | |
| yKA TD ABS | E | 0.535 ± 2.563 | 28 | 0.482 | E | 0.706 ± 2.624 | 25 | 0.478 |
| | R | 0.013 ± 2.752 | 72 | | R | 0.335 ± 2.758 | 61 | |
| xHA TD | E | 25.112 ± 3.132 | 29 | 0.005 | E | 27.946 ± 2.985 | 27 | 0.006 |
| | R | 28.504 ± 6.113 | 73 | | R | 31.068 ± 6.288 | 62 | |
| HW | E | 0.474 ± 0.112 | 29 | 0.005 | E | 0.678 ± 0.142 | 27 | 0.02 |
| | R | 0.409 ± 0.097 | 71 | | R | 0.604 ± 0.130 | 59 | |

Pelvis, Trunk and Spine Angles

| | | | | | | | | |
|---|---|---|---|---|---|---|---|---|
| ΔzPA | E | 11.344 ± 3.044 | 29 | 0.017 | E | 13.403 ± 3.550 | 27 | 0.019 |
| | R | 13.473 ± 4.353 | 73 | | R | 15.665 ± 5.202 | 62 | |
| xPA MAX | E | 1.399 ± 4.073 | 29 | 0.064 | E | 0.192 ± 3.961 | 27 | 0.374 |
| | R | −0.318 ± 4.253 | 73 | | R | −0.670 ± 4.302 | 62 | |
| xPA MIN | E | −7.635 ± 3.332 | 29 | 0.018 | E | −9.113 ± 3.112 | 27 | 0.035 |
| | R | −9.905 ± 4.573 | 73 | | R | 11.090 ± 4.652 | 62 | |
| xPA MEAN | E | −3.023 ± 3.519 | 29 | 0.023 | E | −4.201 ± 3.337 | 27 | 0.095 |
| | R | −5.066 ± 4.193 | 73 | | R | −5.695 ± 4.203 | 62 | |
| ΔxPA | E | 9.034 ± 2.493 | 29 | 0.34 | E | 9.305 ± 2.621 | 27 | 0.046 |
| | R | 9.587 ± 2.422 | 73 | | R | 10.420 ± 2.768 | 62 | |
| ΔyPA | E | 9.489 ± 3.174 | 29 | 0.217 | E | 10.696 ± 3.258 | 27 | 0.134 |
| | R | 10.293 ± 2.692 | 73 | | R | 11.628 ± 3.148 | 52 | |
| Δ zTA | E | 20.073 ± 5.097 | 29 | 0.004 | E | 22.977 ± 5.736 | 27 | 0.002 |
| | R | 23.709 ± 6.484 | 73 | | R | 26.565 ± 6.860 | 62 | |
| LSA MAX | E | 4.308 ± 5.309 | 23 | 0.097 | E | 4.119 ± 5.362 | 21 | 0.015 |
| | R | 6.697 ± 5.722 | 60 | | R | 7.619 ± 6.238 | 44 | |
| Δ LSA | E | 5.214 ± 2.619 | 23 | 0.009 | E | 5.988 ± 3.038 | 21 | 0.004 |
| | R | 7.105 ± 3.352 | 60 | | R | 8.074 ± 4.173 | 44 | |

### The Relationship Between Kinematic Variables and Running Performance, Economy and Coach Ratings.

### Running Economy

Ten kinematic variables were significantly correlated with the energy cost of running across the 3 cohorts (males, females, combined; Table 7), showing weak to moderate correlations (r=0.26 to r=0.57). Several of these variables were quite consistently correlated across the 7 measurements of energy cost (at different speeds and within different cohorts): significantly so for all 7 measurements (VyP^{MIN}), 6 measurements (ΔzP_{H} GC, xHA_{TD}, xKA MIN.GC) or 5 measurements (xHA MAX.SW, SL_{H}). Furthermore, within the larger and more statistically powerful, combined cohort the same 9 variables were correlated with energy cost at both slow and medium speeds. For males the strongest correlates of energy cost were VyP^{MIN}, ΔzP_{H} GC and xKA MIN.GC. For females the strongest correlates of energy cost were ΔzP_{H} GC and xKA MIN.GC. In the more comprehensive combined cohort across the two speeds VyP MIN, ΔzP_{H} GC and xKA MIN.GC were the strongest correlates with r>0.40 for both speeds. The regression analysis for the energy cost of running was able to explain a remarkable 28-43% of the variation in the energy cost of running (across speeds and cohorts; Table 7). Within all 3 cohorts there seemed to be a consistent pattern of more variance explained at slow speeds, likely because the number of runners included is reduced at higher speeds. Also it is the slowest runners (who may have the worst technique) who are no longer included in the analysis at higher speeds likely reducing the variability in the data. The variables contributing to the greatest explained variance was largely due to 3 variables (VyP MIN, ΔzP_{H} GC and xKA MIN.GC). Two of these variables are very similar and likely explain overlapping proportions of the variance: vertical oscillation of the pelvis during ground contact (ΔzP_{H} GC) and maximum flexion of the knee during ground contact (xKA MIN.GC). In this way if one of these variables is very slightly stronger than the second then it takes precedence, and their order seems to change for the different Ec measurements and thus their contribution to the total variation alternates. Qualitatively these two variables might be considered together.

In a simpler approach, when two (VyP MIN, ΔzP_{H} GC) or 3 (VyP MIN, ΔzP_{H} GC, ΔzPA) pelvis variables were forced into the regression analysis then they explained 17-37 or 20-39% of the variance in energy cost, respectively, across all speeds and cohorts (Table 8).

TABLE 6
Significant correlation coefficients between running economy (Energy cost,
Ec) and kinematic variables measured simultaneously at slow (9, 10 & 11 km/hr),
medium (12 & 13 km/hr) and fast (14 & 15 km/hr) running speeds.

| | Males | | | Females | | Combined | |
|---|---|---|---|---|---|---|---|
| | Slow | Medium | Fast | Slow | Medium | Stow | Medium |
| Kinematic Variable: | (n ≥ 47) | (n ≥ 45) | (n ≥ 35) | (n ≥ 45) | (n ≥ 35) | (n ≥ 92) | (n ≥ 80) |

Pelvis & trunk:

| | | | | | | | |
|---|---|---|---|---|---|---|---|
| VyP MIN | −0.54 | −0.45 | −0.46 | −0.44 | −0.36 | −0.50 | −0.40 |
| ΔzP_{H} GC | 0.57 | 0.50 | | 0.55 | 0.39 | 0.56 | 0.41 |
| ΔzPA | 0.33 | 0.45 | | | | 0.28 | 0.39 |
| ΔxTA | | 0.30 | 0.43 | | | 0.31 | 0.26 |

Lower limb position:

| | | | | | | | |
|---|---|---|---|---|---|---|---|
| xHA TD | 0.40 | 0.37 | | 0.34 | 0.44 | 0.34 | 0.39 |
| ΔxHA | 0.36 | 0.31 | | | | | |
| xHA MAX.SW | 0.45 | 0.36 | 0.44 | | | 0.35 | 0.30 |
| xKA MIN.GC | −0.50 | −0.47 | | −0.47 | −0.55 | −0.47 | −0.51 |
| xKA MIN.SW | −0.39 | | | −0.45 | −0.39 | −0.41 | −0.32 |

Stride:

| | | | | | |
|---|---|---|---|---|---|
| SL_{H} | 0.51 | 0.38 | 0.45 | 0.44 | 0.35 |

TABLE 7
Multiple regression analysis for running economy (Energy cost, ec) showing the kinematic variables that
in combination explained the greatest proportion of the variance at slow (9, 10 & 11 km/hr), medium
(12 & 13 km/hr) and fast (14 & 15 km/hr) running speeds within male, female and combined cohorts.

| Variance Explained by Each Kinematic Variable (%) | | | | | | Total Variance |
|---|---|---|---|---|---|---|
| ΔzP_{H} GC | xKA MIN.GC | VyP MIN | ΔzPA | xHA TD | xKA MIN.SW | Explained (%) |

Males:

| | | | | | | |
|---|---|---|---|---|---|---|
| Slow (n ≥ 47) | 33 | 6 | | 4 | | 43 |
| Medium (n ≥ 45) | 25 | 4 | 9 | | | 38 |
| Fast (n ≥ 35) | | 21 | | | 7 | 28 |

Females:

| | | | | |
|---|---|---|---|---|
| Slow (n ≥ 45) | 30 | 8 | 3 | 41 |
| Medium (n ≥ 35) | 4 | 28 | | 32 |

Combined:

| | | | | | |
|---|---|---|---|---|---|
| Slow (n ≥ 92) | 31 | 6 | 4 | | 41 |
| Medium (n ≥ 80) | | 24 | 5 | 3 | 32 |

TABLE 8
Multiple regression analysis for running economy (Energy cost,
EC) at slow (9, 10 & 11 km/hr), medium (12 & 13 km/hr) and fast
(14 & 15 km/hr) running speeds using forced entry of two or three
pelvis variables within male, female and combined cohorts.

| | Total Variance Explained by combined Pelvis Variables (%) | |
|---|---|---|
| Energy | VyP MIN & | VyP MIN, |
| Cost | ΔzP_{H} GC | ΔzP_{H} GC & ΔzPA |
| Males: | | |
| Slow (n ≥ 47) | 37 | 39 |
| Medium (n ≥ 45) | 29 | 38 |
| Fast (n ≥ 35) | 21 | 22 |
| Females: | | |
| Slow (n ≥ 45) | 32 | 32 |
| Medium (n ≥ 35) | 17 | 20 |
| Combined: | | |
| Slow (n ≥ 92) | 35 | 36 |
| Medium (n ≥ 80) | 20 | 28 |

### Running Performance

Fifteen kinematic variables were significantly correlated with measures of running performance across the 3 cohorts (males, females, combined; Table 9), showing weak to moderate correlations (r=0.24 to r=0.59). Of the two measures of performance, vLTP and 10k time, in general the findings were clearer for vLTP, likely due to the fact that this was a standardised laboratory measure of performance made on the same day and under identical conditions to the kinematic measurements. In contrast 10k time was calculated as an equivalence based on each runner's best performance over distances ranging from 1,500 m to the marathon i.e. not the same for all participants, and potentially including different courses (e.g. surfaces, hills and weather conditions etc.). Finally these performances were achieved at any time in the previous 12 months and many factors may have changed prior to the lab visit.

vLTP was correlated with all 7 measurements of axial rotation of the trunk (ΔzTA) across velocity and cohorts, and 6 out of 7 measurements of GCT, xFA TD, ΔzPA and VyP MIN. In the more comprehensive combined cohort the same 12 variables were significant correlates of vLTPz whether measured at slow or medium speeds. The two strongest variables at slow running velocities were VyP MIN (r=0.40) and ΔzP_{H} GC (r=−0.37). At medium speeds the strongest variable was GCT (r=−0.37).

Using multiple regression to explain the greatest proportion of the variance possible, the kinematic variables together explained 29 to 46% of the variance in vLTP, with a pattern for less of the variance to be explained at higher speeds as the weaker runners were excluded from the analysis (Table 10). However, there was considerable inconsistency in the variables that contributed to the total variance explained with this approach. This is almost certainly because of the overlapping nature of the variance explained by many of the variables. For example ground contact time, foot angle at touchdown and shank angle at touchdown are strongly inter-related, and therefore in combination they contribute to the explained variance for all speeds and cohorts, but which variable makes the biggest contribution differs.

In a simpler analysis, when two (VyP MIN, ΔzPA) or three (VyP MIN, ΔzPA & GCT) variables were forced into the regression analysis, they explained 15-25 or 26-38% of the variance in vLTP, respectively, across all speeds and cohorts (Table 11).

For 10k time the findings were less clear, presumably because of the issues outlined above. No single variable was consistently correlated with 10k time across all 5 measurements. For example VyP MIN was not related to 10k time, and GCT was only correlated with 10k time for 3 out of 5 measurements. Therefore no further regression analysis was conducted with 10k time.

TABLE 9
Significant correlation coefficients between running performance and kinematic variables measured
at slow (9, 10 & 11 km/hr), medium (12 & 13 km/hr) and fast (14 & 15 km/hr) running speeds.

| Performance measure: | | | | | | | | | | | |
|---|---|---|---|---|---|---|---|---|---|---|---|
| VLTP | | | | | | | 10k time | | | | |
| Cohort: | | | | | | | | | | | |
| Males (vLTP) | | | Females (vLTP) | | Combined (vLTPz) | | Males | | | Females | |
| Speed: | | | | | | | | | | | |
| Slow | Medium | Fast | Slow | Medium | Slow | Medium | Slow | Medium | Fast | Slow | Medium |
| (n ≥ | (n ≥ | (n ≥ | (n ≥ | (n ≥ | (n ≥ | (n ≥ | (n ≥ | (n ≥ | (n ≥ | (n ≥ | (n ≥ |
| 47) | 45) | 35) | 45) | 35) | 92) | 80) | 47) | 45) | 35) | 45) | 35) |

Pelvis & trunk:

| | | | | | | | | | | | | |
|---|---|---|---|---|---|---|---|---|---|---|---|---|
| VyCM | −0.38 | | | | | −0.3 | −0.24 | 0.59 | 0.35 | | | |
| MAX | | | | | | | | | | | | |
| VyP | −0.43 | −0.36 | −0.39 | 0.36 | | 0.4 | 0.31 | | | | | |
| MIN | | | | | | | | | | | | |
| ΔzP_{H} | | | | −0.42 | | −0.37 | −0.32 | | | | | |
| GC | | | | | | | | | | | | |
| ΔzTA | −0.36 | −0.49 | −0.41 | −0.36 | | −0.31 | −0.24 | 0.29 | 0.42 | 0.44 | | |
| ΔzPA | −0.38 | −0.34 | | −0.36 | −0.4 | −0.35 | −0.31 | | | | | |
| LSA | | | | | | | | | 0.38 | 0.37 | | |
| MAX | | | | | | | | | | | | |
| xTA | | | | | | | | | | | −0.31 | −0.38 |
| MAX | | | | | | | | | | | | |
| xTA | | | | | | 0.28 | 0.29 | | | | | |
| MIN | | | | | | | | | | | | |

Lower limb:

| | | | | | | | | | | | |
|---|---|---|---|---|---|---|---|---|---|---|---|
| GCT | −0.35 | −0.33 | −0.36 | | −0.34 | −0.31 | −0.37 | | 0.3 | 0.38 | 0.44 |
| xFA | −0.38 | −0.3 | | −0.3 | −0.35 | −0.34 | −0.31 | 0.33 | | 0.37 | 0.42 |
| TD | | | | | | | | | | | |
| xSA | −0.36 | −0.31 | | −0.36 | | −0.34 | −0.27 | 0.34 | 0.32 | 0.32 | |
| TD | | | | | | | | | | | |
| ΔxSA | −0.3 | −0.3 | | | | | | 0.34 | 0.35 | 0.35 | |
| GC | | | | | | | | | | | |
| xKA | 0.33 | | | | | 0.28 | 0.3 | | | | −0.4 |
| MIN | | | | | | | | | | | |
| GC | | | | | | | | | | | |
| xHA | | | | −0.39 | −0.5 | −0.32 | −0.3 | | | 0.33 | 0.4 |
| TD | | | | | | | | | | | |
| HW | | | | 0.31 | 0.37 | 0.26 | 0.3 | | | −0.37 | −0.36 |

vLTP, velocity of lactate turnpoint.
vLTPz, velocity of lactate turnpoint expressed as a sex independent z-score.

TABLE 10
Multiple regression analysis for running performance (vLTP/vLTPz) showing the kinematic variables that
in combination explained the greatest proportion of the variance at slow (9, 10 & 11 km/hr), medium
(12 & 13 km/hr) and fast (14 & 15 km/hr) running speeds within male, female and combined cohorts.

| | Variance Explained by Each Kinematic Variable (%) | | | | | | | | | Total Variance |
|---|---|---|---|---|---|---|---|---|---|---|
| vLTP/vLTPz | VyP MIN | ΔzPA | xSA TD | GCT | xFA TD | ΔzTA | HW | xHA TD | ΔzP_{H} GC | Explained (%) |

Males:

| | | | | | | | |
|---|---|---|---|---|---|---|---|
| Slow (n ≥ 47) | 18 | 7 | | | 20 | | 46 |
| Medium (n ≥ 45) | 6 | | 12 | | | 24 | 42 |
| Fast (n ≥ 35) | 10 | | | 10 | | 20 | 40 |

Females:

| | | | | | | | |
|---|---|---|---|---|---|---|---|
| Slow (n ≥ 45) | 7 | 12 | | | | 18 | 37 |
| Medium (n ≥ 35) | 4 | | 25 | 7 | 25 | | 37 |

Combined:

| | | | | | | |
|---|---|---|---|---|---|---|
| Slow (n ≥ 92) | 15 | 3 | 15 | 4 | 7 | 44 |
| Medium (n ≥ 80) | 8 | | | 14 | 7 | 29 |

TABLE 11
Multiple regression analysis for running performance (vLTP/vLTPz)
at slow (9, 10 & 11 km/hr), medium (12 & 13 km/hr) and fast
(14 & 15 km/hr) running speeds using forced entry of two or
three variables within male, female and combined cohorts.

| | Total Variance Explained by Variables (%) | |
|---|---|---|
| vLTP/vLTPz | VyP MIN & ΔzPA | VyP MIN, ΔzPA & GCT |
| Males: | | |
| Slow (n ≥ 47) | 25 | 38 |
| Medium (n ≥ 45) | 21 | 35 |
| Fast (n ≥ 35) | 19 | 36 |
| Females: | | |
| Slow (n ≥ 45) | 20 | 29 |
| Medium (n ≥ 35) | 16 | 26 |
| Combined: | | |
| Slow (n ≥ 92) | 22 | 34 |
| Medium (n ≥ 80) | 15 | 28 |

### Specific Results for the Kinematic Hypotheses and Other Selected Variables of Interest (Study 1 and Study 2)

### Ground Contact Time (GCT)

Study 1—Recreational males had a significantly longer ground contact time (GCT) than elite males at 11 and 13 km·h⁻¹ (P=0.004 and 0.007 respectively). Similarly for the combined data recreational runners had a significantly longer GCT than elites at 9, 11 and 13 km·h⁻¹ (P=0.007, 0.002 and 0.003 respectively). However, there were no significant differences between groups for the females only. GCT significantly correlated with performance measures for the males only (vLPT) at 11, 13 and 15 km·h⁻¹, and the combined data (IAAF points) at 9, 11 and 13 km·h⁻¹, but not for the females only. There were no significant correlations between GCT and running economy.

Study 2—Both recreational males and females had significantly longer ground contact times (GCT) than the respective elite groups at the slow and medium speeds (P=0.03 and 0.02 respectively for the males; P=0.02 and P=0.02 respectively for the females). No differences were observed at the fast speed. GCT was significantly correlated with performance measures at a number of speeds for the males and females, but not with energy cost. A longer GCT at the slow and medium speeds was correlated with a longer 10k time in both sexes. A longer GCT at the slow, medium and fast speeds for males, and at the slow and medium speeds for females, was correlated with a lower vLTP.

### Ground Contact Distance, Normalised to Height (GCD_{H})

Study 1—Recreational males had a significantly greater ground contact distance (GCD_{H}) than elite males at 11 and 13 km·h⁻¹ (P=0.001 and 0.004 respectively). Similarly for the combined data recreational runners had a significantly greater GCD_{H} than elites at 9, 11 and 13 km·h⁻¹ (P=0.008, 0.002 and 0.004 respectively). However, there were no significant differences between groups for the females. GCD_{H} significantly correlated with performance measures for the males only (10k time and vLPT) at 11, 13 and 15 km·h⁻¹, but not for the females only or the combined data. There were no significant correlations between GCD_{H} and running economy.

Study 2—Recreational males had a significantly greater ground contact distance (GCD_{H}) than elite males at the slow, medium and fast speeds (P=0.01, P=0.01 and 0.04 respectively). Recreational females had a significantly greater GCD_{H} than elite females at the slow and medium speeds (P=0.03 and P=0.02), but no differences were observed at the fast speed. GCD_{H} was significantly correlated with performance measures at a number of speeds for the males and females, but not with energy cost. A greater GCD_{H} at the slow, medium and fast speeds for males, and the slow and medium speeds for females, was correlated with an increased 10k time. A greater GCD_{H} at all three speeds was also correlated with a lower vLTP for males, but this relationship was only seen at the medium speed in females.

### Touchdown to Centre of Mass Distance, Normalised to Height (TD-CM_{H})

Study 1—Recreational males had a significantly greater touchdown to centre of mass (horizontal) distance (TD-CM_{H}) than elite males at 11 and 13 km·h⁻¹ (P=0.008 and 0.024 respectively). Similarly for the combined data recreational runners had a significantly longer GCD than elites at 9, 11 and 13 km·h⁻¹ (P=0.037, 0.017 and 0.026 respectively). However, there were no significant differences between groups for the females. TD-CM_{H} significantly correlated with performance measures for the males only (10k time and vLPT) at 11, 13 and 15 km·h⁻¹, but not for the females only or the combined data. There were no significant correlations between TD-CM_{H} and running economy.

Study 2—Recreational males had a significantly greater touchdown to centre of mass distance (TD-CM_{H}) than elite males at the slow and medium speeds (P=0.03 and P=0.02), but no differences were observed at the fast speed. No differences were observed between the elite and recreational females at any speed. TD-CM_{H} was significantly correlated with just a few measures. A greater TD-CM_{H} at the medium speed was correlated with increased energy cost in females, but no relationships were observed for the males. A greater TD-CM_{H} at the slow speed was correlated with an increased 10k time for males, but no relationships observed for females. A greater TD-CM_{H} at the slow and medium speeds was correlated with a lower vLTP, but no relationships were observed in the females.

### Toe-Off to Centre of Mass Distance, Normalised to Height (TO-CM_{H})

Study 1—Recreational males had a significantly greater toe-off to centre of mass (horizontal) distance (TO-CM_{H}) than elite males at 11, 13 and 15 km·h⁻¹ (P=0.012, 0.004 and 0.029 respectively). Similarly for the combined data recreational runners had a significantly greater TO-CM_{H} than elites at 9, 11 and 13 km·h⁻¹ (P=0.008, 0.002 and 0.004 respectively). However, there were no significant differences between groups for the females. TO-CM_{H} significantly correlated with performance measures for the males only (10k time and vLPT) at 11, 13 and 15 km·h⁻¹, and for the combined data (IAAF points and vLTPz) at 9 and 13 km·h⁻¹, but not for the females only. There were no significant correlations between TO-CM_{H} and running economy.

Study 2—Recreational males had a significantly greater toe-off to centre of mass distance (TO-CM_{H}) than elite males at the slow, medium and fast speeds (P=0.04, P=0.01 and P=0.03). Recreational females had a significantly greater TO-CM_{H} than elite females at the slow and medium speeds (P=0.02 and P=0.01), but no differences were observed at the fast speed. TO-CM_{H} was significantly correlated with performance measures at a number of speeds for the males and females, but not with energy cost. An increase in TO-CM_{H} at all speeds was correlated with an increased 10K time in both males and females. An increase in TO-CM_{H} at the medium and high speeds for males, and at the medium speed for females was correlated with a lower vLTP.

### Duty Factor (DF)

Study 1: Recreational males had a significantly greater duty factor (DF) than elite males at 11 and 13 km·h−1 (P=0.013 and 0.013 respectively). Similarly for the combined data recreational runners had a significantly greater DF than elites at 11 and 13 km·h⁻¹ (P=0.007 and 0.002 respectively). There were no significant differences between groups for the females only. DF significantly correlated with performance measures for the males only (10k time) at 15 km·h⁻¹, but not for the females only or combined data. DF significantly correlated with running economy for the females only at 9 km·h⁻¹, and the combined data at 9 and 11 km·h⁻¹, but not for the males only.

Study 2: Recreational males had a significantly greater duty factor (DF) than elite males at the medium and fast speeds (P=0.02 and P=0.03), but no differences were observed at the fast speed. Females had a significantly greater DF at the medium speed (P=0.02), but no differences were observed at the slow or fast speeds. An increase in DF at the medium and high speeds in males, and the medium speed in females, was correlated with an increased 10k time.

Change in Vertical Position of the Centre of Mass, Normalised to Height (ΔzCM_{H})

Study 1: There were no significant differences between recreational and elite runners in the vertical displacement of the centre of mass (ΔzCM). ΔzCM significantly correlated with performance measures for the females only (10k time and vLPT) and the combined data (vLTPz) at 9 km·h⁻¹ only, but not for the males only. ΔzCM significantly correlated with running economy for the males only, females only and the combined data up to 11 km·h⁻¹.

Study 2: There were no significant differences between recreational and elite runners in the vertical displacement of the centre of mass during each whole step (ΔzCM_{H}). An increase in ΔzCM_{H} at the slow and medium speeds in males, and the slow speed in females, was correlated with an increased energy cost.

### Change in Velocity of the Whole Body Centre of Mass (ΔVyCM)

Study 1: In the combined data, recreational runners had a greater change in the horizontal velocity of the centre of mass during ground contact (ΔVyCM) than the elites at 9 km·h⁻¹ only (P=0.042). There were no significant differences between groups for the males only or females only. ΔVyCM significantly correlated with performance measures for the males only (vLTP) at 11 km·h⁻¹ and the combined data (IAAF points and vLTPz) at 9 km·h⁻¹. ΔVyCM significantly correlated with running economy for the males only at 11, 13 and 15 km·h⁻¹ and the combined data at 11 and 15 km·h⁻¹. There were no significant correlations with performance or running economy for the females only.

Study 2: There were no significant differences between recreational and elite runners in change in velocity of the whole body centre of mass during ground contact, ΔVyCM.

An increase in ΔVyCM at the slow speed was correlated with a longer 10k time, lower vLTP and an increased energy cost in males, but no relationships were observed for females. An increase in ΔVyCM at the medium speed was also related to a lower vLTP, but no further relationships were observed.

### Mean Anterior Lean of the Pelvis (xPA MEAN)

Study 1: Recreational females had significantly greater mean anterior tilt of the pelvis (xPA MEAN) than the elites at 9, 11 and 13 km·h⁻¹ (P=0.001, 0.003 and 0.009 respectively). Similarly for the combined data recreational runners had significantly greater xPA MEAN than the elites at 9 km·h⁻¹ (P=0.021) only. However, there were no significant differences between groups for the males only. xPA MEAN significantly correlated with performance measures for the females only (10k time and vLTP) at 9, 11 and 13 km·h⁻¹ and the combined data (vLTPz) at 9 km·hr⁻¹. xPA MEAN significantly correlated with running economy for the combined data at 9 km·h⁻¹ only. There were no significant correlations with performance or running economy for the males only.

Study 2: Recreational females had a significantly greater mean anterior lean (more negative) of the pelvis (xPA MEAN) than elite females at the slow, medium and fast speeds (P=0.04, and P=0.01 and P=0.03). No differences were observed between the elite and recreational groups at any speed in the males. A more negative xPA MEAN at the slow, medium and fast speeds was correlated with a lower vLTP in the females. A more negative xPA MEAN at the slow speed for males, and at the medium speed for females, was correlated with an increased energy cost. No relationship between xPA MEAN and 10k was observed.
Change in Axial Angle of the Pelvis (ΔzPA) (Hypothesis 5d)

Study 1: In the combined data, recreational runners had a greater axial rotation of the pelvis (ΔzPA) than the elites at 13 km·h⁻¹ only (P=0.038). There were no significant differences between groups for the males only or females only. ΔzPA significantly correlated with performance measures for the females only (10k time and vLTP) at 9, 11 and 13 km·h⁻¹ and the combined data (vLTPz) at 9 and 11 km·hr−1 but not for the males only. ΔzPA significantly correlated with running economy for the males only at 11 km·h⁻¹ and both the females only and combined data at 9, 11 and 13 km·h⁻¹.

Study 2: Recreational females displayed a greater range of motion in axial rotation angle of the pelvis (ΔzPA) than elite females at the slow and medium speeds (P=0.02 and P=0.02). No differences between recreational and elite males were observed. An increased ΔzPA at the slow and medium speeds for males, and the slow, medium and fast speeds for females was correlated with a lower vLTP. An increased ΔzPA at the slow and medium speed was correlated with an increased energy cost. An increased ΔzPA at the medium speed was also correlated with an increased 10k time, but only in females.

Mean Vertical Position of the Pelvis, Normalised to Height (zP̅_{H})

Study 1: There were no significant differences between recreational and elite runners in the mean anterior tilt of the trunk (zP̅_{H}). There were no significant correlations between zP̅_{H} and measures of performance. However, zP̅_{H} significantly correlated with running economy for the females only and the combined data at 9 and 11 km·h⁻¹, but not for the males only.

Change in vertical position of the pelvis, normalised to height (ΔzP_{H})

Study 1: There were no significant differences between recreational and elite runners in the change in vertical position of the pelvis (ΔzP_{H}). ΔzP_{H} significantly correlated with performance measures for the females only (10k time and vLTP) and for the combined data (vLTPz) at 9 and 11 km·h⁻¹, but not for the males only. There were no significant correlations with running economy.

Study 2: An increased ΔzPH at the slow speed for males, and the slow and fast speeds for females, was correlated with a lower vLTP. An increased ΔzPH at the slow and medium speeds for the males, and at the slow speed for the females, was correlated with an increased energy cost. No correlations between ΔzPH and 10 k time were observed.

Minimum horizontal velocity of the pelvis (VyP^{MIN})

Study 1: Recreational females had significantly lower minimum horizontal velocity of the pelvis (VyP^{MIN}) than the elites 11 km·h⁻¹ (P=0.031). Similarly for the combined data recreational runners had significantly lower VyP^{MIN} than the elites at 11 km·h⁻¹ (P=0.048). However, there were no significant differences between groups for the males only. VyP^{MIN} significantly correlated with performance measures for the males only (vLTP), the females only (10k time and vLTP) and the combined data (vLTPz) across velocities. Similarly, VyP^{MIN} significantly correlated with running economy for the males only, females only and the combined data across velocities.

Study 2: A lower VyP^{MIN} at all speeds for the males, and the slow and fast speeds for females, was correlated with a lower vLTP. A lower VyP^{MIN} at all speeds for the males, and the slow and medium speeds for females, was correlated with an increased energy cost. No correlations between VyP^{MIN} and 10 k time were observed.

### Change in Horizontal Velocity of the Pelvis (ΔVyP)

Study 1: There were no significant differences between recreational and elite runners in the change in pelvis horizontal velocity during ground contact (ΔVyP). ΔVyP significantly correlated with performance measures for the males only (vLTP), the females only (10k time and vLTP) and the combined data (vLTPz) at 9 and 11 km·h⁻¹. Similarly, ΔVyP significantly correlated with running economy for the males only, the females only (10k time and vLTP) and the combined data (vLTPz) across velocities.

Study 2: An increased ΔVyP at the slow and fast speeds for males was correlated with a lower vLTP, no relationships were observed in the females. An increased ΔVyP at all speeds for the males, and the slow and medium speeds for females, was correlated with an increased energy cost.

### Mean Lower Spine Angle (LSA MEAN)

Study 1: In the combined data, recreational runners had a greater mean lower spine angle (L̅S̅A̅) than the elites at 13 km·h⁻¹ only (P=0.040). There were no significant differences between groups for the males only or females only. L̅S̅A̅ significantly correlated with performance measures for the males only (10k time and vLPT) and the combined data (IAAF points and vLTPz) at 11 and 13 km·h⁻¹, but not for the females only. L̅S̅A̅ did not significantly correlate with running economy.

Study 2: Recreational males displayed a significantly higher mean lower spine angle (LSA MEAN) at all three speeds, than elite males (P=<0.05, P=0.02, P=0.04). An increased LSA MEAN at the medium and fast speeds in males was correlated with an increased 10k time. No further correlations were observed.

### Hip Work Done (HW)

Study 1: Recreational runners did significantly less positive work at the hip during swing (HW) than the elites at 9 and 13 km·h⁻¹ for the females only (P=0.029 and 0.035 respectively) and the combined data (P=0.001, 0.010 and 0.043 respectively). However, there were no significant differences between groups for the males only. HW significantly correlated with performance measures for the females only (10k time and vLPT) and the combined data (IAAF points and vLTPz) at 9 km·h⁻¹, but not for the males only. HW did not significantly correlate with running economy.

Study 2: The recreational females displayed a significantly lower amount of positive work done at the hip (HW) than the elite females at the slow and medium speeds (P=0.01 and P=0.03) but not at the fast speed. No between group differences were observed for the males. An increased HW at the slow and medium speeds for females was correlated with a lower vLTP.

### Sagittal Angle of Foot at Touch-Down (xFA TD)

Study 2: Both recreational males and females displayed significantly larger sagittal plane angles of the foot at touch-down (xFA TD) than the respective elite groups at the slow and medium speeds (P=0.01 and 0.04 respectively for the males; P=0.04 and P=0.03 respectively for the females). No differences were observed at the fast speed. An increase in xFA TD at the slow speed in males, and the slow and medium speeds in females, was correlated with an increased 10k time. No correlations between xFA TD and energy cost or vLTP were observed.

### Minimum Knee Flexion During Ground Contact (xKA MIN.GC)

Study 2: Recreational females had a significantly greater knee flexion (more negative) during ground contact (xKA MIN.GC) than the elite females at the slow and medium speeds (P=0.04 and P=0.03 respectively). No differences were observed at the fast speed in females, or any of the speeds between the male groups.

A more negative xKA MIN.GC at the slow speed for males, and the slow and medium speeds for females, was correlated with an increased 10k time. A more negative xKA MIN.GC at the slow speed for both sexes was also correlated with an increased energy cost.

### Hip Flexion at Touch Down (xHA TD)

Study 2: Recreational females had a significantly greater hip flexion at touch down (xHA TD) than the elite females at all speeds (P=0.00, P=0.00 and P=0.00 respectively). No differences were observed between the male groups.

A greater xHA TD at all speeds in females was correlated with an increased 10k time and a lower vLTP. xHA TD was not correlated with 10k time or vLTP in males. A greater xHA TD at the slow and medium speeds was correlated with an increased energy cost.

Change in Anterior Angle of the Pelvis (ΔxPA)

Study 2—Recreational males had a significantly greater range of motion in anterior pelvis angle (ΔxPA) than elite males at the medium and fast speeds (P=0.02, and P=0.02), but no differences were observed at the slow speed. No differences were observed between the elite and recreational females at any of the speeds.

A greater ΔxPA at the medium and fast speeds was correlated with an increased 10k time in the males, but no relationships were observed for the females. A greater ΔxPA at the medium speed was correlated with a lower vLTP in males, but no relationships were observed for the females. No relationship between ΔxPA and energy cost was observed for either sex.

Range of Motion in Axial Rotation Angle of the Trunk (ΔzTA)

Study 2—Recreational females displayed a significantly greater range of motion in axial rotation angle of the trunk (ΔzTA) than the elite group at the slow, medium and fast speeds (P=0.02, P=0.03 and P=0.00 for females). The recreational males displayed a significantly greater (ΔzTA) than the elite males at the medium and fast speeds (P=0.03 and P==0.04) but not at the slow speed.

An increase in ΔzTA at the slow speed in both males and females was correlated with an increased 10k time. An increased ΔzTA at all the speeds for males, and at the slow and fast speeds for females, was correlated with a lower vLTP. No correlations between ΔzTA and energy cost were observed.

### Detailed Explanation of the Kinematic Variables

FIG. 5 shows an example of touchdown and toe-off identification. FIG. 5 (a) Vertical acceleration of the right heel and first metatarsal head markers were used to identify right foot touchdown. The acceleration peaks for each marker occurring immediately after the change in anterior-posterior velocity of the heel marker (from positive to negative, not shown) were initially identified (shown by the solid circles and squares). The peak that occurred first was then set as touchdown (RTD, solid vertical black lines); in this case the heel marker (solid line and solid circle). FIG. 5 (b) Vertical jerk of the right toe marker was used to identify right foot take-off. The jerk peak occurring in a fixed time window after touchdown was identified (black circle) and then set as take-off (RTO, dashed vertical black lines). Note that the solid and dashed vertical grey lines are the left foot touchdown and take-offs as identified by a similar process carried out on the left heel, first metatarsal head and toe markers. Ground contact time is preferably calculated using the timing of the vertical acceleration peak of either the heel or metatarsal markers, whichever occurs first, for touchdown and the timing of the vertical jerk peak of the toe marker for toe-off. This enables the accurate measurement of GCT across a range of running speeds and footstrike types.

FIG. 6 shows an example of centre of mass vertical movement and anterior-posterior velocity measurement. FIG. 6 (a) Vertical position of the centre of mass. The maximum and minimum were calculated for each step (the region between vertical line RTD to adjacent vertical line LTD show the right steps, i.e. right foot touchdown to left foot touchdown) and then the range determined as the difference between the maximum and minimum on a step-by-step basis. The horizontal dashed line represents the centre of mass vertical position during quiet standing. FIG. 6 (b) Anterior-posterior velocity of the centre of mass. The maximum and minimum were calculated for each ground contact phase (region between solid RTD line to adjacent dashed vertical line show the right foot ground contacts). The maximum was constrained to occur after the minimum and up to take-off.

FIG. 7 shows an example of pelvis vertical position and anterior-posterior velocity measurement. FIG. 7 (a) Vertical position of the pelvis. The maximum and minimum were calculated for each step (region from solid RTD vertical line to next LTD vertical line show the right steps, i.e. right foot touchdown to left foot touchdown) and then the range determined as the difference between the maximum and minimum on a step-by-step basis. The horizontal dashed line represents the pelvis vertical position during quiet standing. The solid horizontal line represents the mean vertical pelvis position during ground contact only. FIG. 7 (b) Anterior-posterior velocity of the pelvis. The maximum and minimum were calculated for each ground contact phase (region within RTD vertical line to the adjacent dashed vertical line show the right foot ground contacts). The maximum was constrained to occur after the minimum and up to take-off.

FIG. 8 shows an example of pelvic rotation angle measurement. FIG. 8 (a) Pelvis tilt. The maximum and minimum were calculated for each stride (region between RTD solid vertical line to next RTD solid vertical line show the strides defined as right foot touchdown to the next right foot touchdown) and then the range determined as the difference between the maximum and minimum on a stride-by-stride basis. The horizontal dashed line represents the pelvis tilt during quiet standing. The solid horizontal line represents the mean pelvis tilt during the entire stride. FIG. 8 (b) Pelvis obliquity. The maximum and minimum were calculated for each stride and then the range determined as the difference between the maximum and minimum on a stride-by-stride basis. The horizontal dashed line represents the pelvis obliquity during quiet standing. FIG. 8 (c) Pelvis axial rotation. The maximum and minimum were calculated for each stride and then the range determined as the difference between the maximum and minimum on a stride-by-stride basis. The horizontal dashed line represents the pelvis axial rotation angle during quiet standing

FIG. 9 shows an example of trunk rotation angle measurement. FIG. 9 (a) Trunk tilt. The maximum and minimum were calculated for each stride (the region between left hand vertical RTD line and next vertical solid RTD line show the strides defined as right foot touchdown to the next right foot touchdown) and then the range determined as the difference between the maximum and minimum on a stride-by-stride basis. The horizontal dashed line represents the trunk tilt during quiet standing. The solid horizontal line represents the mean trunk tilt during the entire stride. FIG. 9 (b) Trunk axial rotation. The maximum and minimum were calculated for each stride and then the range determined as the difference between the maximum and minimum on a stride-by-stride basis. The horizontal dashed line represents the trunk axial rotation angle during quiet standing.

FIG. 10 shows an example of Lower limb flexion-extension angle measurement. FIG. 10(a) Hip flexion-extension. The touchdown and take-off values were determined for each ground contact phase (region between RTD vertical line and adjacent dashed vertical line show the right foot ground contacts). The horizontal dashed line represents the hip flexion-extension angle during quiet standing. FIG. 10(b) Knee flexion-extension. The touchdown and take-off values and the minimum knee (flexion) angle were determined for each ground contact phase. The horizontal dashed line represents the knee flexion-extension angle during quiet standing. FIG. 10(c) Ankle dorsiflexion-plantar flexion. The touchdown and take-off values and the maximum ankle (dorsiflexion) angle were determined for each ground contact phase. The horizontal dashed line represents the ankle dorsiflexion-plantar flexion angle during quiet standing.

FIG. 11 shows an example of the measurement of foot and shank angles to the vertical. FIG. 11 (a) the touchdown and take-off values were determined for each ground contact phase (region between RTD vertical line and adjacent dashed vertical line show the right foot ground contacts). The horizontal dashed line represents the shank angle during quiet standing. FIG. 11 (b) Foot angle. The touchdown and take-off values were determined for each ground contact phase. The horizontal dashed line represents the foot angle during quiet standing.

FIG. 12 shows an example of the measurement of upper and lower sagittal plane spine angles. FIG. 12 (a) Upper spine angle. The maximum and minimum were calculated for each stride (region between RTD vertical line to next RTD vertical line show the strides defined as right foot touchdown to the next right foot touchdown) and then the range determined as the difference between the maximum and minimum on a stride-by-stride basis. The horizontal dashed line represents the upper spine angle during quiet standing. The solid horizontal line represents the mean upper spine angle during the entire stride. FIG. 12 (b) Lower spine angle. The maximum and minimum were calculated for each stride and then the range determined as the difference between the maximum and minimum on a stride-by-stride basis. The horizontal dashed line represents the lower spine angle during quiet standing. The solid horizontal line represents the mean lower spine angle during the entire stride.

Other variations and modifications will be apparent to the skilled person. Such variations and modifications may involve equivalent and other features that are already known and which may be used instead of, or in addition to, features described herein. Features that are described in the context of separate embodiments may be provided in combination in a single embodiment. Conversely, features that are described in the context of a single embodiment may also be provided separately or in any suitable sub-combination.

It should be noted that the term “comprising” does not exclude other elements or steps, the term “a” or “an” does not exclude a plurality, a single feature may fulfil the functions of several features recited in the claims and reference signs in the claims shall not be construed as limiting the scope of the claims. It should also be noted that the Figures are not necessarily to scale; emphasis instead generally being placed upon illustrating the principles of the present invention.

What is claimed:

1. A system for monitoring the running technique of a user undertaking a physical activity, the system comprising:
at least one garment worn by the user, the garment incorporating at least one sensor for the detection of at least one parameter relating to the motion of the user wherein at least one sensor detects at least one parameter relating to the movement of the pelvis of the user;
a processing unit configured to receive information about the at least one parameter from the at least one sensor, to compare the or each parameter with at least one aspect of a biomechanical model of the physical activity, and to determine if a feedback response is required; and means for providing the feedback response to the user.

2. A system according to claim 1 wherein the feedback response is provided to the user during the physical activity.

3. A system according to claim 1 in which the means for providing a feedback response to the user comprises at least one haptic actuator, preferably wherein the haptic actuator is embedded in at least one garment worn by the user.

4. A system according to claim 1 wherein at least one sensor detects the minimum forward pelvic velocity of the user.

5. A system according to claim 1 wherein the comparison with the biomechanical model comprises analysis of one or more of (i) the velocity of the pelvis; (ii) the change in vertical position of the pelvis; (iii) the axial rotation of the pelvis; (iv) the anterior angle of the pelvis.

6. A system according to claim 1 wherein the comparison with the biomechanical model comprises analysis of one or more of the following (i) the velocity of the pelvis and ground contact time; (ii) the velocity of the pelvis and the change in vertical position of the pelvis; (iii) the velocity of the pelvis and the axial rotation of the pelvis; (iv) the velocity of the pelvis and change in velocity of the centre of mass of the user.

7. A system according to claim 1 wherein the comparison with the biomechanical model comprises analysis of one or more of the following (i) the velocity of the pelvis, ground contact time, and the axial rotation of the pelvis; (ii) the velocity of the pelvis, the axial rotation of the pelvis and the change in vertical position of the pelvis.

8. A system according to claim 1 wherein the sensor detects at least one parameter relating to the ground contact of the user, wherein the comparison with the biomechanical model comprises analysis of one or more of (i) ground contact time; (ii) flight time; (iii) duty factor: (iv) touchdown to centre of mass distance; (v) take-off to centre of mass distance; (vi) ground contact distance.

9. (canceled)

10. A system according to claim 1 wherein the sensor detects at least one parameter relating to the stride pattern of the user, wherein the comparison with the biomechanical model comprises analysis of one or more of (i) stride rate: (ii) stride Length.

11. (canceled)

12. A system according to claim 1 wherein the sensor detects at least one parameter relating to the centre of mass of the user, wherein the comparison with the biomechanical model comprises analysis of one or more of (i) change in velocity of the centre of mass of the user; (ii) change in vertical position of the centre of mass of the user.

13. (canceled)

14. A method for monitoring the running technique of an individual undertaking an physical activity, the method comprising the steps of:
(i) measuring at least one parameter relating to the motion of the individual, wherein the at least one parameter relates to the movement of the pelvis of the individual;
(ii) comparing the or each parameter with at least one aspect of a biomechanical model of running to determine whether a feedback response is required;
(iv) providing a feedback response to the individual.

15. A method according to claim 14 wherein at least one parameter is measured with at least one sensor incorporated within a garment worn by the individual.

16. A method according to claim 14 wherein a feedback response is provided during the physical activity.

17. A method according to claim 14 in which the feedback response is provided by at least one haptic actuator embedded in a garment worn by the individual.

18. A method according to claim 14 wherein the method comprises the measurement of the minimum forward pelvic velocity of the individual.

19. A method according to claim 14 wherein the comparison with the biomechanical model comprises analysis of one or more of (i) the velocity of the pelvis; (ii) the change in vertical position of the pelvis; (iii) the axial rotation of the pelvis; (iv) the anterior angle of the pelvis.

20. A method according to claim 14 wherein the method comprises the measurement of at least one parameter relating to the ground contact of the individual, wherein the comparison with the biomechanical model comprises analysis of one or more of (i) ground contact time; (ii) flight time; (iii) duty factor; (iv) touchdown to centre of mass distance; (v) take-off to centre of mass distance; (vi) ground contact distance.

21. (canceled)

22. A method according to claim 14 wherein the method comprises the measurement of at least one parameter relating to the stride pattern of the individual, wherein the comparison with the biomechanical model comprises analysis of one or of (i) stride rat (ii) stride length.

23. (canceled)

24. A method according to claim 14 wherein the method comprises the measurement of at least one parameter relating to the centre of mass of the individual, wherein the comparison with the biomechanical model comprises analysis of one or more of (i) change in velocity of the centre of mass of the individual; (ii) change in vertical position of the centre of mass of the individual.

25. (canceled)

26. A method according to claim 14 wherein the comparison with the biomechanical model comprises analysis of one or more of the following (i) the velocity of the pelvis and ground contact time; (ii) the velocity of the pelvis and the change in vertical position of the pelvis; (iii) the velocity of the pelvis and the axial rotation of the pelvis; (iv) the velocity of the pelvis and change in velocity of the centre of mass of the user; (v) the velocity of the pelvis, ground contact time, and the axial rotation of the pelvis; (vi) the velocity of the pelvis, the axial rotation of the pelvis and the change in vertical position of the pelvis.
